(19) United States
(12) Patent Application Publication
(10) Pub. No.: US 2009/0162295 A1
(43) Pub. Date: Jun. 25, 2009

(54) SETTING POLYMERS BASED ON POLYESTER ACRYLATES

(75) Inventors: Gabi Winter, Wachenheim (DE); Marianna Pierobon, Ludwigshafen (DE); Matthias Laubender, Schifferstadt (DE); Son Nguyen Kim, Hemsbach (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 12/063,511

(22) PCT Filed: Aug. 2, 2006
(86) PCT No.: PCT/EP2006/064960

§ 371 Date: Sep. 29, 2008

(30) Foreign Application Priority Data
Aug. 11, 2005 (EP) 05107394.8

(51) Int. Cl. A61K 9/12; C08F 220/26; A61K 47/32

(52) U.S. Cl. 424/45; 526/320; 526/318.4; 514/772.6

(57) ABSTRACT

The present invention relates to cosmetic preparations which comprise at least one polymer A which comprises, in copolymerized form, at least one ester of (meth)acrylic acid, at least one olefinically unsaturated anionogenic or anionic compound, at least one polyester or polyether with at least two free-radically polymerizable, olefinically unsaturated double bonds, and if appropriate further olefinically unsaturated compounds.

The present invention relates to cosmetic preparations which comprise at least one polymer A which comprises, in copolymerized form, 40-89.5% by weight of at least one ester of (meth)acrylic acid, 10-49% by weight of at least one olefinically unsaturated anionogenic or anionic compound, 0.5-10% by weight of at least one polyester or polyether with at least two free-radically polymerizable, olefinically unsaturated double bonds, and, if appropriate, 0-30% by weight of further olefinically unsaturated compounds.

### PRIOR ART

Stricter environmental regulations and a growing ecological awareness increasingly demand ever lower fractions of volatile organic components (VOCs) in cosmetic aerosol preparations such as, for example, aerosol hairsprays.

The VOC content in hairsprays is essentially determined by the nonaqueous solvents and the propellants. For this reason, instead of nonaqueous solvents, recourse is currently and increasingly being made to water as solvent. However, this replacement of the organic solvents has a number of problems.

Thus, formulations of the film-forming polymers known from the prior art which satisfy the corresponding VOC regulations are not, for example, sprayable, or are only sprayable following further dilution and are thus only of limited suitability for use in hairsprays. Polymer films which are formed from such preparations sometimes do not have the required mechanical quality and thus inadequate setting effect and poor hold for the hair.

### OBJECT AND SOLUTION

One object of the present invention was to provide polymers for cosmetic, in particular hair cosmetic, preparations which can readily be formulated as pump or aerosol spray in solvents or solvent mixtures with an increased water fraction, whose formulations are readily sprayable in the form of small uniform droplets and, during and after application, have the lowest possible tendency for foaming and whose films then formed are not sticky and have good mechanical properties.

Besides the good compatibility with the customary cosmetic ingredients, the polymers applied to the hair should dry rapidly and impart good setting and prolonged hold to the hair even at increased atmospheric humidity, have a good ability to be washed out and be able to be formulated as optically clear VOC 55 aerosols (i.e. with a VOC fraction of at most 55% by weight). In addition, the treated hair should have good haptic properties such as, for example, a good feel to the touch.

Surprisingly, these objects were achieved by cosmetic preparations comprising at least one polymer A which comprises in copolymerized form,
a) 40-89.5% by weight of at least one ester of (meth)acrylic acid,
b) 10-49% by weight of at least one olefinically unsaturated, anionogenic or anionic compound,
c) 0.5-10% by weight of at least one compound chosen from
   c1) polyesters comprising at least two free-radically polymerizable, olefinically unsaturated double bonds and
   c2) polyethers comprising at least two free-radically polymerizable, olefinically unsaturated double bonds,
d) 0-30% by weight of, if appropriate, further olefinically unsaturated compounds
   with the proviso that the amounts of components a) to d) add up to 100% by weight.

WO 03/062288 and WO 03/061615 describe aqueous hair-setting compositions which comprise an effective amount of a rheology-modifying hair-setting associative polymer from an acid monomer and an associative monomer. Preferred hydrophobic associative monomers are long-chain esters of (meth)acrylic acid. The polymers can comprise further monomers and crosslinkers.

EP-A 0 184 785 describes an aqueous copolymer dispersion of 50-60% by weight of ethyl acrylate, 30-40% by weight of methacrylic acid, 5-15% by weight of acrylic acid and 0.02-0.04% by weight of a polyunsaturated copolymerizable monomer with a solids content of 5-30% by weight which is suitable for thickening aqueous systems, in particular hydrogen peroxide preparations, as are used as developer preparations for oxidation hair colorants and for hair bleaches. Polyunsaturated monomers are only used in very small amounts.

WO 95/05402 describes hair cosmetic preparations which comprise aqueous copolymer dispersions obtainable by copolymerization of from 40 to 99% by weight of one or more water-insoluble, monoethylenically unsaturated monomers and 1 to 60% by weight of one or more water-soluble, monoethylenically unsaturated monomers. Optionally, 0 to 30% by weight of one or more ethylenically polyunsaturated monomers can be used.

DE 2 330 957 describes grafted and crosslinked cationic copolymers obtained by copolymerization of a) at least one cosmetic monomer, b) dimethylaminoethyl methacrylate, c) polyethylene glycol and d) a polyunsaturated crosslinker.

U.S. Pat. No. 3,940,351 describes polymers of ethylenically unsaturated carboxylic acids, long-chain esters of such carboxylic acids and polyethers comprising allyl groups, the preparation of which is carried out in the presence of a haloalkane. Within the scope of the present invention, the expression alkyl comprises straight-chain and branched alkyl groups. Suitable short-chain alkyl groups are, for example, straight-chain or branched C₁-C₁₂-alkyl, preferably C₁-C₆-alkyl and particularly preferably C₁-C₄-alkyl groups. These include, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethyl-propyl, 2,2-dimethylpropyl, 1-ethylpropyl, n-hexyl, 2-hexyl, 2-methylpentyl, 3-methyl-pentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc.

Suitable longer-chain C₈-C₃₀-alkyl or C₈-C₃₀-alkenyl groups are straight-chain and branched alkyl or alkenyl groups. These are preferably predominantly linear alkyl radicals as also arise in natural or synthetic fatty acids and fatty alcohols and also oxo alcohols, which may, if appropriate, additionally be mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene) etc.

Cycloalkyl is preferably C₅-C₈-cycloalkyl, such as cyclopentyl, cyclohexyl, cycloheptyl or cyclooctyl.

For the purposes of the present invention, the expression heterocycloalkyl comprises saturated, cycloaliphatic groups having generally 4 to 7, preferably 5 or 6, ring atoms, in which 1 or 2 of the ring carbon atoms are replaced by heteroatoms chosen from the elements oxygen, nitrogen and sulfur and which may, if appropriate, be substituted, where in the case of a substitution, these heterocycloaliphatic groups can carry 1, 2 or 3, preferably 1 or 2, particularly preferably 1, substituent chosen from alkyl, aryl, COOR, COO⁻M⁺ and NE¹E², preferably alkyl. Examples of such heterocycloaliphatic groups which may be mentioned are pyrrolidinyl, piperidinyl, 2,2,6,6-tetramethyl-piperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

Aryl comprises unsubstituted and substituted aryl groups and is preferably phenyl, tolyl, xylyl, mesityl, naphthyl, fluorenyl, anthracenyl, phenanthrenyl, naphthacenyl and, in particular, phenyl, tolyl, xylyl or mesityl.

Substituted aryl radicals preferably have 1, 2, 3, 4 or 5, in particular 1, 2 or 3, substituents chosen from alkyl, alkoxy, carboxyl, carboxylate, trifluoromethyl, —SO₃H, sulfonate, NE¹E², alkylene-NE¹E², nitro, cyano or halogen.

Hetaryl is preferably pyrrolyl, pyrazolyl, imidazolyl, indolyl, carbazolyl, pyridyl, quinolinyl, acridinyl, pyridazinyl, pyrimidinyl or pyrazinyl.

Arylalkyl is groups which comprise both alkyl and aryl radicals, these arylalkyl groups being joined to the compound carrying them via the aryl radical or via the alkyl radical.

### Component a)

Component a) is chosen from the esters of (meth)acrylic acid.

Component a) is chosen, for example, from the group consisting of methyl (meth)acrylate, ethyl (meth)acrylate, n-propyl (meth)acrylate, isopropyl (meth)acrylate, n-butyl (meth)acrylate, tert-butyl (meth)acrylate, isobutyl (meth)acrylate, sec-butyl (meth)acrylate, 2-pentyl (meth)acrylate, 3-pentyl (meth)acrylate, isopentyl (meth)acrylate, neopentyl (meth)acrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arrachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acyrlate, stearyl (meth)acrylate, lauryl (meth)acrylate, phenoxyethyl (meth)acrylate, 4-t-butylcyclohexyl acrylate, cyclohexyl (meth)acrylate, ureido (meth)acrylate, tetrahydrofurfuryl (meth)acrylate and mixtures thereof.

Component a) can also be chosen from esters of (meth)acrylic acid with alkanediols. These are, for example, 2-hydroxyethyl (meth)acrylate, 2-hydroxyethyl ethacrylate, 2-hydroxypropyl (meth)acrylate, 3-hydroxypropyl (meth)acrylate, 3-hydroxybutyl (meth)acrylate, 4-hydroxybutyl (meth)acrylate, 6-hydroxyhexyl (meth)acrylate, 3-hydroxy-2-ethylhexyl (meth)acrylate, neopentyl glycol mono(meth)acrylate, 1,5-pentanediol mono(meth)acrylate and 1,6-hexanediol mono(meth)acrylate.

Preferred (meth)acrylates are C₁-C₁₀-, particularly preferably C₁-C₈- and in particular C₁-C₄-alkyl (meth)acrylates. Component a) can also be a mixture of methacrylates and acrylates.

Component a) is very particularly preferably chosen from the group consisting of tert-butyl acrylate, methyl methacrylate, ethyl methacrylate, n-propyl methacrylate, isopropyl methacrylate, n-butyl methacrylate, tert-butyl methacrylate, isobutyl methacrylate, sec-butyl methacrylate and mixtures thereof.

Component a) is still further preferably chosen from the group consisting of methyl methacrylate, ethyl methacrylate and mixtures thereof.

Component a) is most preferably methyl methacrylate (MMA).

Polymer A comprises 40-89.5, particularly preferably 60-80 and in particular 70-80% by weight of component a) in copolymerized form.

### Component b)

Component b) is an olefinically unsaturated, free-radically polymerizable anionogenic or anionic compound. Within the scope of the present invention, an anionogenic compound is understood as meaning a compound which can be converted into the corresponding anionic form by deprotonation with customary, preferably cosmetically acceptable, organic or inorganic bases.

Component b) can be chosen from olefinically unsaturated, free-radically polymerizable carboxylic acids, sulfonic acids or phosphonic acids and organic and inorganic salts thereof.

Examples of preferred sulfonic acids are 2-acrylamido-2-methylpropanesulfonic acid (AMPS), styrenesulfonic acid, vinylsulfonic acid and salts thereof.

Examples of preferred phosphonic acids are vinylphosphonic acid, 2-acrylamido-2-methylpropanephosphonic acid, allylphosphonic acid and salts thereof.

Component b) is preferably chosen from the group of olefinically unsaturated, free-radically polymerizable carboxylic acids and organic and inorganic salts thereof. The carboxylic acids are monocarboxylic acids, dicarboxylic acids, carboxylic anhydrides or half-esters of dicarboxylic acids.

Component b) is particularly preferably chosen from the group consisting of acrylic acid, methacrylic acid, ethacrylic acid, alpha-chloroacrylic acid, crotonic acid, maleic acid, maleic anhydride, itaconic acid, citraconic acid, mesaconic acid, glutaconic acid, aconitic acid, fumaric acid, half-esters of olefinically unsaturated dicarboxylic acids having 4 to 10, preferably 4 to 6, carbon atoms and salts thereof.

Component b) comprises or consists very particularly preferably of compounds chosen from the group consisting of acrylic acid, methacrylic acid, salts thereof and mixtures thereof.

Component b) comprises or consists very particularly preferably of compounds chosen from the group consisting of acrylic acid, itaconic acid, salts thereof and mixtures thereof.

Polymer A comprises 10-49, particularly preferably 12-39 and in particular 15-29% by weight of component b) in copolymerized form.

### Component c)

Component c) is chosen from

c1) polyesters comprising at least two free-radically polymerizable, olefinically unsaturated double bonds and
c2) polyethers comprising at least two free-radically polymerizable, olefinically unsaturated double bonds.

Polyesters and polyethers are known in principle to the person skilled in the art. Preferred polyesters c1) and polyethers c2) are polyester and polyether (meth)acrylates. Within the scope of this invention, polyester and polyether (meth)acrylates are the terms used for compounds which, in addition to the (meth)acrylate ester groups present, comprise at least two further, preferably more than two further, ester and/or ether groups.

Of course, the polyesters can also comprise ether structural units and the polyethers can also comprise ester structural units. Numerous suitable components c) comprise both ester and ether groups at the same time. Component c) of course also comprises any mixtures of c1) and c2).

Preferred components c) comprise, as one of the at least two free-radically polymerizable, olefinically unsaturated double bonds, a (meth)acrylate group of the general formula H₂C═CR—COO—, where R is H or methyl. Further preferred components c) comprise at least two (meth)acrylate groups.

Component c1)

The term polyester is known to the person skilled in the art. Polyesters are polymers with ester bonds —[—CO—O—]— in the main chain. Components c1) according to this invention are, for example, polyester (meth)acrylates which comprise at least two free-radically polymerizable, olefinically unsaturated double bonds per molecule.

Polyester (meth)acrylates are known in principle to the person skilled in the art. They can be prepared by various methods. For example, (meth)acrylic acid can be used directly as acid component when building up the polyesters. In addition, there is the option of using hydroxyalkyl esters of (meth)acrylic acid as alcohol component directly when building up the polyesters. The polyester (meth)acrylates are, however, preferably prepared by (meth)acrylation of polyesters. For example, polyesters containing hydroxyl groups can firstly be built up, which are then reacted with acrylic or methacrylic acid. Preferably, at least two of the hydroxyl groups are reacted with (meth)acrylic acid per molecule of the polyesters containing hydroxyl groups, meaning that per molecule of the reaction product, at least two free-radically polymerizable, olefinically unsaturated double bonds are present.

It is also possible to firstly build up polyesters containing carboxyl groups, which are then reacted with a hydroxyalkyl ester of acrylic or methacrylic acid. Here too, at least two of the carboxyl groups are reacted with the hydroxyalkyl ester of (meth)acrylic acid per molecule of the polyesters containing carboxyl groups, meaning that per molecule of the reaction product at least two free-radically polymerizable, olefinically unsaturated double bonds are present.

It is preferred to use mixtures of polyester (meth)acrylates which comprise, on average, more than two free-radically polymerizable, olefinically unsaturated double bonds per molecule of polyester (meth)acrylate.

Polyester acrylates suitable as component c1) are described, for example, in EP-A 0 279 303, which is hereby incorporated in its entirety by reference (EP-A 0 279 303, p. 5, II.28-44).

DE 2 853 921 also describes suitable polyester acrylates, namely those of aliphatic and/or aromatic dicarboxylic acids, such as succinic acid, glutaric acid, adipic acid, pimelic acid, suberic acid, azelaic acid, sebacic acid, cyclohexanedicarboxylic acid, phthalic acid, isophthalic acid, terephthalic acid, maleic acid, fumaric acid, itaconic acid and derivatives thereof

and polyhydric alcohols, such as ethylene glycol, polyethylene glycols, propylene glycol, polypropylene glycols, butanediol, hexanediol, neopentyl glycol, neopentyl glycol hydroxypivalate, trimethylolpropane, glycerol, pentaerythritol and/or trishydroxyethyl isocyanurate
and α,β-ethylenically unsaturated monocarboxylic acids, for example acrylic acid, methacrylic acid, crotonic acid, cinnamic acid and/or dicarboxylic half-esters of monoalkanols, such as maleic, fumaric and itaconic half-esters with C₁-C₄-monoalcohols, with acrylic acid and methacrylic acid being preferred.

EP-A 0 686 621, which is hereby incorporated in its entirety by reference, also describes suitable components c1). These are reaction products of (meth)acrylic acid with a hydroxy compound. Suitable hydroxy compounds are compounds with one or more hydroxy groups.

Monoalcohols, C₂-C₆-alkylenediols, trimethylolpropane, glycerol or pentaerythritol or compounds comprising hydroxy groups alkoxylated, for example, with ethylene oxide or propylene oxide are specified.

Suitable hydroxy compounds are also polyesters which contain hydroxyl groups. Such polyesters containing hydroxyl groups can be prepared, for example, in the usual manner by esterification of dicarboxylic acids or polycarboxylic acids with diols or polyols. The starting materials for such polyesters containing hydroxyl groups are known to the person skilled in the art. Dicarboxylic acids which can be used are preferably succinic acid, glutaric acid, adipic acid, sebacic acid, o-phthalic acid, isomers thereof and hydrogenation products, and also esterifiable derivatives, such as anhydrides, e.g. maleic anhydride, or dialkyl esters of the specified acids. Suitable polycarboxylic acids are, for example, trimellitic acid. The polyesterols which can be used also include polycaprolactonediols and -triols, the preparation of which is likewise known to the person skilled in the art.

Preferred hydroxy compounds are saturated polyesters comprising at least 2, in particular 2 to 6, free hydroxyl groups, which can, if appropriate, also comprise ether groups, or polyethers (as component c2)) with at least 2, in particular 2 to 6, free hydroxyl groups.

The components c1), such as, for example, polyester (meth)acrylates, have at least 2 free-radically polymerizable double bonds per molecule. It is also preferred to use mixtures of components c), for example of polyester (meth)acrylates which comprise, on average, more than 2 free-radically polymerizable, olefinically unsaturated double bonds per molecule of polyester (meth)acrylate. Such mixtures arise, for example, by mixing compounds each with 2 and compounds each with 3 or more polymerizable double bonds per molecule. It is of course also possible for compounds which comprise only one or no double bond per molecule to also be present in the mixtures. However, such compounds are then present in amounts such that the average number of polymerizable double bonds per molecule is nevertheless more than two.

Component c2)

The term polyether is known to the person skilled in the art. Polyethers are polymers whose organic repeat units are held together by ether functionalities (C—O—C). Examples of polyethers are polyalkylene glycols (polyethylene glycols, polypropylene glycols, polyepichlorohydrins) as polymers of 1,2-epoxides, epoxy resins, polytetrahydrofurans (polytetramethylene glycols), polyoxetanes, polyphenylene ethers (polyaryl ethers) or polyether (ether) ketone (ketone)s.

Components c2) according to this invention are, for example, polyether (meth)acrylates which comprise at least two free-radically polymerizable double bonds per molecule. These are likewise known in principle to the person skilled in the art. They can be prepared by various methods. For example, polyethers containing hydroxyl groups and which are esterified with acrylic acid and/or methacrylic acid to give the polyether (meth)acrylates can be obtained by reacting di- and/or polyhydric alcohols with different amounts of ethylene oxide and/or propylene oxide in accordance with well known methods (cf. e.g. Houben-Weyl, Volume XIV, 2, Makromolekulare Stoffe II, (1963)). It is also possible to use polymerization products of tetrahydrofuran or butylene oxide.

DE 2 853 921, which is hereby incorporated in its entirety by reference, also describes suitable components c2), such as, for example, aliphatic or aromatic-aliphatic polyethers which are obtained by reacting di- and/or polyhydric alcohols with different amounts of ethylene oxide and/or propylene oxide and whose free-hydroxyl groups are completely or partially etherified with ethylenically unsaturated alcohols, for example alkyl alcohol, methallyl alcohol, crotyl alcohol, cinnamyl alcohol and/or esterified with α,β-ethylenically unsaturated monocarboxylic acids.

Polyether acrylates suitable as component c2) are described, for example, in EP-A 0 279 303, which is hereby incorporated in its entirety by reference.

These polyether acrylates are obtainable by reacting A) one equivalent of a 2- to 6-hydric oxalkylated C₂- to C₁₀-alcohol with B) 0.05 to 1 equivalent of a 2- to 4-basic C₂- to C₁₀-carboxylic acid or anhydrides thereof and C) 0.1 to 1.5 equivalents of acrylic acid and/or methacrylic acid, and reacting the excess carboxyl groups with the equivalent amount of an epoxide compound.

EP-A 0 686 621, which is hereby incorporated in its entirety by reference, also describes suitable components c2). These are reaction products of (meth)acrylic acid with a hydroxy compound. Suitable hydroxy compounds are compounds with one or more hydroxy groups. Mention may be made, for example, of compounds comprising hydroxy groups which are alkoxylated with ethylene oxide or propylene oxide.

Preferred hydroxy compounds are saturated polyethers with at least 2, in particular 2 to 6, free hydroxyl groups. Suitable polyethers containing hydroxyl groups are, for example, those which can be obtained by known processes by reacting di- and/or polyhydric alcohols with varying amounts of ethylene oxide and/or propylene oxide. In the case of the ethylene glycol/propylene glycol co-condensation products, the reaction can expediently be controlled so that ultimately predominantly primary hydroxyl groups form. It is likewise also possible to use polymerization products of tetrahydrofuran or butylene oxide which comprise hydroxyl groups.

Examples of component c) are polyalkylene glycol (meth)acrylates.

In a preferred embodiment of the invention, compounds used as component c) are those whose molecular weight M_{w} is at least 200 g/mol, particularly preferably at least 400 g/mol, very particularly preferably at least 500 g/mol and most preferably more than 700 g/mol.

In a further preferred embodiment of the invention, compounds c1) and/or c2) or mixtures of compounds c1) and/or c2) are used as component c), where the average number of olefinic, free-radically polymerizable double bonds per molecule is more than 2. Such mixtures arise, for example, by mixing compounds each with 2 and compounds each with 3 or more polymerizable double bonds per molecule. It is of course also possible for compounds which comprise only one or no double bond per molecule to also be present in mixtures. However, such compounds are then present in amounts such that the average number of polymerizable double bonds per molecule is nevertheless more than 2.

It should at this point be emphasized that there are compounds suitable as component c) which can be allotted to both groups c1) and c2) since they comprise both ester groups and ether groups. Component c) according to the invention are thus also compounds comprising at least two free-radically polymerizable, olefinically unsaturated double bonds which comprise both ether structures and also ester structures at the same time.

Commercially available products which are suitable as component c) are, for example:

Photomer®5010, Photomer®5429, Photomer®5430, Photomer®5432, Photomer®5662, Photomer®5806, Photomer®5930 from Cognis;
the Resin® grades from UCB, such as, for example, Resin®80, 81, 83, 450, 657, 770, 809, 810, 830, 835, 870, 1657, 1810, 1870, 2047**, 2870;
the CN® grades from Sartomer such as, for example, CN293, CN294, CN296, CN292, CN2297A, CN2279, CN2280, CN2470, CN295, CN2300, CN2200, CN2203, CN2282, CN2284, CN2270, CN2271, CN2272, CN2273, CN2276, CN2250, CN2251, CN2252, CN2253, CN2255, CN2256, CN2257, CN2258, CN2259, CN2260, CN2261;
AROPLAZ®4097-WG4-55 from Reichhold;
Syntholux®-PE grades from Synthopol as polyester acrylates and the Syntholux®-PA grades from Synthopol as polyether acrylates;
Laromer® grades Laromer®PE 55F, Laromer® PE 56F, Laromer®PE 46T, Laromer®9004, Laromer®PE 44F, Laromer®8800, Laromer®LR 8981, Laromer®LR 8992, Laromer®PE 22WN, Laromer®PE 55WN, Laromer®PO 33F, Laromer®LR 8863, Laromer®PO 43F, Laromer®LR 8967, Laromer®LR 8982, Laromer®LR 9007 (BASF).

Polymer A comprises 0.5-10, preferably 1-8, particularly preferably 1-5% by weight of component c) in copolymerized form.

### Component d)

Suitable components d) are all free-radically polymerizable, unsaturated compounds which are different from the components a) to c) and which can be copolymerized with components a) to c).

Preferred components d) are

d1) compounds containing amide groups different from d2),
d2) (meth)acrylamides,
d3) cationogenic monomers,
d4) cationic monomers,
d5) compounds with at least two polymerizable double bonds, which are usually also referred to as crosslinkers and
d6) mixtures thereof.
Component d1)

The compounds d1) containing amide groups are preferably chosen from compounds which are different from d2) and of the general formula VI

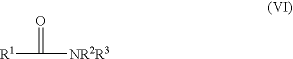

where R¹ is a group of the formula CH₂═CR⁴— where R⁴═H or C₁-C₄-alkyl and R² and R³, independently of one another, are H, alkyl, cycloalkyl, heterocycloalkyl, aryl or hetaryl or R² and R³ together with the nitrogen atom to which they are bonded are a five- to eight-membered nitrogen heterocycle or
R² is a group of the formula CH₂═CR⁴— and R¹ and R³, independently of one another, are H, alkyl, cycloalkyl, heterocycloalkyl, aryl or hetaryl or R¹ and R³ together with the amide group to which they are bonded are a lactam having 5 to 8 ring atoms.

Preferred components d1) are N-vinyllactams. Suitable components d1) are unsubstituted N-vinyllactams and N-vinyllactam derivatives, which can, for example, have one or more C₁-C₆-alkyl substituents, such as methyl, ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc. and mixtures thereof.

Preferred components d1) are those for which, in formula VI, R² is CH₂═CH— and R¹ and R³ together with the amide group to which they are bonded are a lactam having 5 ring atoms.

Particular preference is given to using N-vinylpyrrolidone, N-vinylcaprolactam, N-vinylformamide, acrylamide or mixtures thereof, with N-vinylpyrrolidone being most preferred.

Component d2)

Suitable components d2) are the amides of (meth)acrylic acid different from d3) and d4). Such amides are, for example, (meth)acrylamide, N-methyl(meth)acrylamide, N-ethyl(meth)acrylamide, N-n-propyl(meth)acrylamide, N-i-propyl(meth)acrylamide, N-(n-butyl)methacrylamide, N-(sec-butyl)methacrylamide, N-(tert-butyl)methacrylamide, N-(n-pentyl)(meth)acrylamide, N-(n-hexyl)(meth)acrylamide, N-(n-heptyl)(meth)acrylamide, N-(n-octyl)(meth)acrylamide, N-(tert-octyl)(meth)acrylamide N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arrachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide.

Suitable components d2) are also 2-hydroxyethylacrylamide, 2-hydroxyethylmethacrylamide, 2-hydroxyethylethacrylamide, 2-hydroxypropylacrylamide, 2-hydroxypropylmethacrylamide, 3-hydroxypropylacrylamide, 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide.

Components d3) and d4)

The components d3) and d4) are monomers which comprise at least one cationogenic and/or cationic group per molecule.

Preferably, the cationogenic and cationic groups are nitrogen-containing groups, such as primary, secondary and tertiary amino groups, and quaternary ammonium groups. The nitrogen-containing groups are preferably tertiary amino groups.

The components d3) and d4) are preferably used for the polymerization in uncharged form. However, use in charged form is also suitable.

Charged cationic groups can be produced, for example, from the amine nitrogen atoms by protonation, for example with monobasic or polybasic carboxylic acids, such as lactic acid or tartaric acid, or mineral acids, such as phosphoric acid, sulfuric acid and hydrochloric acid.

The components d3) and d4) are preferably chosen from
esters of α,β-olefinically unsaturated mono- and dicarboxylic acids with amino alcohols, which may be mono- or dialkylated on the amine nitrogen,
amides of α,β-olefinically unsaturated mono- and dicarboxylic acids with diamines which have at least one primary or secondary amino group,
N,N-diallylamine,
N,N-diallyl-N-alkylamines and derivatives thereof,
vinyl- and allyl-substituted nitrogen heterocycles
vinyl- and allyl-substituted heteroaromatic compounds and
mixtures thereof.

Suitable components d3) and d4) are also the esters of α,β-olefinically unsaturated mono- and dicarboxylic acids with amino alcohols. Preferred amino alcohols are C₂-C₁₂-aminoalcohols which are C₁-C₈-mono- or -dialkylated on the amine nitrogen. Suitable acid components of these esters are, for example, acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof.

Particularly preferred components d3) and d4) are N-methylaminoethyl (meth)acrylate, N-ethylaminoethyl (meth)acrylate, N-(n-propyl)aminoethyl (meth)acrylate, N-(n-butyl)aminoethyl (meth)acrylate, N-(tert-butyl)aminoethyl (meth)acrylate, N,N-dimethylaminomethyl (meth)acrylate, N,N-dimethylaminoethyl (meth)acrylate, N,N-diethylaminoethyl (meth)acrylate, N,N-dimethylaminopropyl (meth)acrylate, N,N-diethylaminopropyl (meth)acrylate and N,N-dimethylaminocyclohexyl (meth)acrylate.

In particular, the components d3) and d4) used are N-(tert-butyl)aminoethyl acrylate and N-(tert-butyl)aminoethyl methacrylate.

Suitable components d3) and d4) are also the amides of the abovementioned α,β-olefinically unsaturated mono- and dicarboxylic acids with diamines which have at least one primary or secondary amino group.

Preference is given to diamines which have one tertiary and one primary or secondary amino group. As components d3) and d4), preference is given to using N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide and N-[4-(dimethylamino)cyclohexyl]methacrylamide.

Particular preference is given to using N-[3-(dimethylamino)propyl]acrylamide and/or N-[3-(dimethylamino)propyl]methacrylamide.

Suitable components d3) and d4) are also N,N-dialkylamines and N,N-diallyl-N-alkylamines and acid addition salts thereof. Alkyl here is preferably C₁-C₂₄-alkyl. Preference is given to N,N-diallyl-N-methylamine.

Suitable components d3) and d4) are also vinyl- and allyl-substituted nitrogen heterocycles, such as N-vinylimidazole, N-vinylimidazole derivatives, e.g. N-vinyl-2-methylimidazole, vinyl- and allyl-substituted heteroaromatic compounds, such as 2- and 4-vinylpyridine, 2- and 4-allylpyridine, and the salts thereof.

Suitable components d3) and d4) are also N-vinylimidazoles of the general formula VII in which R¹ to R³ are hydrogen, C₁-C₄-alkyl or phenyl

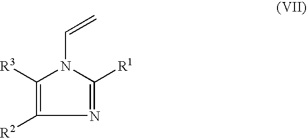

Examples of compounds of the general formula VII are given in table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl;
Ph = phenyl

The components d3) and d4) are particularly preferably chosen from N-(tert-butyl-amino)ethyl (meth)acrylate, N,N-dimethylaminoethyl (meth)acrylate, N-[3-(dimethylamino)propyl](meth)acrylamide, vinylimidazole and mixtures thereof.

If the polymers A according to the invention comprise components d3) and/or d4) in copolymerized form, then they comprise at least 0.1% by weight, preferably at least 1% by weight, particularly preferably at least 2% by weight and in particular at least 3% by weight and at most 30% by weight, preferably at most 20% by weight, particularly preferably at most 15% by weight and in particular at most 10% by weight of the components d3) and/or d4), based on the total weight of the components a) to d) used.

The charged cationic groups can be produced from the amine nitrogens by quaternization with so-called alkylating agents. Examples of suitable alkylating agents are C₁-C₄-alkyl halides or sulfates, such as ethyl chloride, ethyl bromide, methyl chloride, methyl bromide, dimethyl sulfate and diethyl sulfate. A quaternization can generally take place either before or after the polymerization.

Component d5)

Component d5) are compounds with at least two free-radically polymerizable nonconjugated double bonds per molecule.

Suitable components d5) are, for example, acrylates, methacrylates, allyl ethers or vinyl ethers of at least dihydric alcohols. The OH groups of the parent alcohols here may be completely or partially etherified or esterified; however, the components d5) comprise at least two free-radically polymerizable unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, neopentyl glycol monohydroxypivalate, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000.

Apart from the homopolymers of ethylene oxide and propylene oxide, it is also possible to use block copolymers of ethylene oxide or propylene oxide or copolymers which comprise incorporated ethylene oxide and propylene oxide groups.

Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. Preferred polyhydric alcohols in this connection are also di- and trisaccharides.

The polyhydric alcohols can of course also be used following reaction with ethylene oxide or propylene oxide in the form of the corresponding ethoxylates or propoxylates. The polyhydric alcohols can also firstly be converted to the corresponding glycidyl ethers by reaction with epichlorohydrin.

Further suitable components d5) are the vinyl esters or the esters of monohydric, unsaturated alcohols with olefinically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, it is also possible to esterify the monohydric unsaturated alcohols with polybasic carboxylic acids, for example malonic acid, tartaric acid, trimellitic acid, phthalic acid, terephthalic acid, citric acid or succinic acid.

Further suitable components d5) are esters of unsaturated carboxylic acids with the above-described polyhydric alcohols, for example oleic acid, crotonic acid, cinnamic acid or 10-undecanoic acid.

Suitable components d5) are also straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Further suitable components d5) are also the amides of (meth)acrylic acid, itaconic acid and maleic acid, and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids, as have been described above.

Also suitable are triallylamine and triallylmonoalkylammonium salts, e.g. triallylmethylammonium chloride or methyl sulfate, as component d5).

Also suitable are N-vinyl compounds of urea derivatives, at least difunctional amides, cyanurates or urethanes, for example of urea, ethyleneurea, propyleneurea or tartramide, e.g. N,N′-divinylethyleneurea or N,N′-divinylpropyleneurea.

Also suitable as alkylenebisacrylamides, such as methylenebisacrylamide and N,N′-(2,2)butane and 1,1′-bis(3,3′-vinylbenzimidazolith-2-one)-1,4-butane.

Other suitable components d5) are, for example, alkylene glycol di(meth)acrylates, such as ethylene glycol diacrylate, ethylene glycol dimethacrylate, tetraethylene glycol acrylate, tetraethylene glycol dimethacrylate, diethylene glycol acrylate, diethylene glycol methacrylate, vinyl acrylate, allyl acrylate, allyl methacrylate, divinyidioxane, pentaerythritol allyl ether and mixtures of these components d5).

Further suitable components d5) are divinyldioxane, tetraallylsilane or tetravinylsilane.

Particularly preferred components d5) used are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred components d5) are pentaerythritol triallyl ether, methylenebisacrylamide, N,N′-divinylethyleneurea, triallylamine and triallylmonoalkyl-ammonium salts, and acrylic esters of glycol, butanediol, trimethylolpropane or glycerol or acrylic esters of glycol, butanediol, trimethylolpropane and glycerol reacted with ethylene oxide and/or epichlorohydrin.

Particularly preferred component d5) are compounds with a molecular weight M_{w} of <400 g/mol.

Mixtures of the abovementioned compounds can of course also be used. The component d5) is preferably soluble in the reaction medium. If the solubility of component d5) in the reaction medium is low, then it can be dissolved in a monomer or in a monomer mixture, or else be metered in dissolved form in a solvent which is miscible with the reaction medium. Particular preference is given to those components d5) which are soluble in the monomer mixture.

If the component d5) is used to prepare the polymer A) according to the invention, then the amount used is at least 0.01, preferably at least 0.05, particularly preferably at least 0.1 and at most 5, preferably at most 2 and particularly preferably at most 1% by weight, based on the total amount of the components a) to d).

If the polymers A according to the invention are to comprise a component d5) in copolymerized form, then it is particularly advantageous to use mixtures of components c) and d5). Such mixtures are commercially available and, besides the component c), comprise as so-called reactive thinners, for example tripropylene glycol diacrylate (TPGDA), hexanediol diacrylate (HDDA), dipropylene glycol diacrylate (DPGDA), trimethylolpropane formal monoacrylate (e.g. Laromer®LR 8887), trimethylolpropane triacrylate (TMPTA), propoxylated glyceryl triacrylate (GPTA), ethoxylated trimethylolpropane triacrylate (EO3TMPTA), ethoxyethoxyethyl acrylate (EOEOEA), PEG 400 diacrylate (PEG400DA), isobornyl acrylate (IBOA), propoxylated neopentyl glycol diacrylate (PO2NPGDA), 2-phenoxyethyl acrylate (POEA), butanediol diacrylate (BDDA), butanediol acrylate (BDMA), dihydrodicyclopentadienyl acrylate (DCPA), triethylene glycol divinyl ether, ethyl diglycol acrylate (EDGA), lauryl acrylate (LA), 4-t-butylcyclohexyl acrylate (TBCH).

As component d) it is also possible to use vinyl acetate, vinyl propionate, vinyl butyrate, ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The components d) can also comprise silicone-containing structural elements.

Polymer A comprises 0-30, preferably 0-20, particularly preferably 1-15 and in particular 2-10% by weight of component d) in copolymerized form.

The polymers A present in the preparations according to the invention preferably comprise, in copolymerized form,
a) 65-80% by weight of component a),
a) 12-39% by weight of component b),
b) 1-8% by weight of component c) and
c) 0-20% by weight of component d)
   with the proviso that the amounts of components a) to d) add up to 100% by weight.

The polymers A present in the preparations according to the invention particularly preferably comprise, in copolymerized form,
a) 70-80% by weight of component a),
b) 15-29% by weight of component b),
c) 1-5% by weight of component c) and
d) 0-20% by weight of component d)
   with the proviso that the amounts of components a) to d) add up to 100% by weight.

### Preparation of the Polymers a According to the Invention

The polymers A according to the invention can be prepared, for example, by solution polymerization, precipitation polymerization, suspension polymerization or emulsion polymerization. Such processes are customary and known to the person skilled in the art.

The preparation is preferably by solution polymerization. It is preferred to prepare the polymers A by free-radical solution polymerization.

Preferred solvents for the polymerization are alcoholic or alcoholic/aqueous solvents, such as ethanol or isopropanol and mixtures of ethanol or isopropanol with water and/or further alcohols, such as methanol, n-propanol, n-butanol, sec-butanol, tert-butanol, n-hexanol and cyclohexanol, and glycols, such as ethylene glycol, propylene glycol and butylene glycol, and the methyl or ethyl ethers of the dihydric alcohols such as diethylene glycol, triethylene glycol, polyethylene glycols with number-average molecular weights up to about 3000, glycerol and dioxane.

The polymerization is particularly preferably in alcohol, for example in ethanol or in an alcohol/water mixture, for example in an ethanol/water mixture.

The polymerization temperatures are preferably in a range from about 30 to 140° C., particularly preferably 40 to 120° C. The polymerization usually takes place under atmospheric pressure, although it can also proceed under reduced or increased pressure. A suitable pressure range is between 1 and 5 bar.

For the copolymerization, the monomers can be polymerized with the help of initiators which form free radicals.

Initiators which can be used for the free-radical polymerization are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethyl-hexanoate, tert-butyl permaleate, cumene hydroperoxide, diisopropyl peroxy-dicarbamate, bis(o-toloyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, 2,2′-azobis(2-amidinopropane) hydrochloride (Walko V-50®), 2,2′-azobis[2-(2-imidazolin-2-yl)propane] (Wako VA-061®), 2,2′-azobis(2-methylbutyronitrile) (Wako V-59®), dimethyl 2,2′-azobis(2-methylpropionate) (Wako V-601®), 2,2′-azobis(2,4-dimethylvaleronitrile), 1,1′-azobis(1-cyclohexane-carbonitrile), 4,4′-azobis(4-cyanovaleric acid) or 2-(carbamoylazo)isobutyronitrile.

Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{I}, H₂O₂/ascorbic acid.

Suitable oxidizing agents for redox initiator systems are essentially the peroxides given above. Corresponding reducing agents which can be used are sulfur compounds with a low oxidation state, such as alkali metal sulfites, for example potassium and/or sodium sulfite, alkali metal hydrogensulfites, for example potassium and/or sodium hydrogensulfite, alkali metal metabisulfites, for example potassium and/or sodium metabisulfite, formaldehyde sulfoxylates, for example potassium and/or sodium formaldehyde sulfoxylate, alkali metal salts, specifically potassium and/or sodium salts of aliphatic sulfinic acids and alkali metal hydrogensulfides, such as, for example, potassium and/or sodium hydrogensulfide, salts of polyvalent metals, such as iron(II) sulfate, iron(II) ammonium sulfate, iron(II) phosphate, enediols, such as dihydroxy-maleic acid, benzoin and/or ascorbic acid, and reducing saccharides, such as sorbose, glucose, fructose and/or dihydroxyacetone.

It may also be advantageous to use mixtures of water-soluble initiators and initiators which are insoluble or sparingly soluble in water. It may also be advantageous to use mixtures of organic and inorganic initiators.

Suitable initiators are described in chapters 20 and 21 of Macromolecules, Vol. 2, 2nd Ed., H. G. Elias, Plenum Press, 1984, New York, which is hereby incorporated in its entirety by reference. Furthermore, suitable photoinitiators are described in S. P. Pappas, J. Rad. Cur., July 1987, p. 6, which is hereby incorporated in its entirety by reference.

The initiators are usually used in amounts up to 10, preferably 0.02 to 5, % by weight, based on the monomers to be polymerized.

The K value of the polymers is in the range from 15 to 120, preferably from 25 to 75 and particularly preferably from 25 to 55 (determination in accordance with Fikentscher, Cellulosechemie, Vol. 13, p. 58 to 64 (1932)). Ways of adjusting the K value of polymers to a value in a desired range are known to the person skilled in the art. For example, these are the polymerization temperature, the amount of initiator or the use of chain transfer reagents.

To adjust the molecular weight, the polymerization can be carried out in the presence of at least one chain transfer reagent (regulator). Chain transfer reagents which can be used are the customary compounds known to the person skilled in the art, such as, for example, sulfur compounds, e.g. mercaptoethanol, 2-ethylhexyl thioglycolate, thioglycolic acid, alkanethiols, cysteine, acetylcysteine, and tribromochloromethane or other compounds which have a regulating effect on the molecular weight of the polymers obtained.

The regulators are usually used in amounts of from 0.1 to 5% by weight, in particular 0.25 to 2% by weight, based on the monomers to be polymerized. Usually, the regulators are added to the polymerization together with the monomers.

To achieve the purest possible polymers A with a low residual monomer content, the polymerization (main polymerization) can be followed by at least one after-polymerization step. The after-polymerization can take place in the presence of the same initiator system as the main polymerization, or a different initiator system. Preferably, the after-polymerization takes place at least at the same temperature as the main polymerization, preferably at a higher temperature. If desired, following the polymerization or between the first and the second polymerization step, the reaction mixture can be subjected to stripping with water vapor or to a water vapor distillation.

The copolymerization takes place in accordance with the customary processing techniques of solution polymerization, e.g. according to the so-called batch polymerization, in which the monomers and, if appropriate, polymerization regulator and initiator are initially introduced in a solvent and heated to the polymerization temperature. The reaction mixture is preferably stirred at the polymerization temperature until the conversion of the monomers is more than 99.9%. In these processes, the initiators can, if appropriate, also be added once the polymerization temperature has been reached.

Further process variants are feed methods, which are preferably used. In these, individual reaction participants or all of the reaction participants are added, completely or partially, in batches or continuously, together or in separate feeds, to a reaction mixture. Thus, for example, it is possible, for example, if appropriate to add a solution of the polymerization regulator and an initiator solution continuously or batchwise to a mixture of the monomers and of a solvent at the polymerization temperature within a given time. It is also possible to meter a mixture of initiator and, if appropriate, regulator into the initial charge heated to polymerization temperature. Another variant consists in adding the initiator to the initial charge below or at the polymerization temperature and, if a regulator is to be used, to only add the regulator or a solution of the regulator to the reaction mixture within a pregiven time after the polymerization temperature has been reached.

The organic solvent used in the preparation of the polymers can be removed by customary methods known to the person skilled in the art, e.g. by distillation at reduced pressure. The mixtures which form during the polymerization can be subjected to a physical or chemical after-treatment following the polymerization process. Such processes are, for example, the known processes for reducing residual monomers, such as, for example, after-treatment by adding polymerization initiators or mixtures of two or more polymerization initiators at suitable temperatures or heating the polymerization solution to temperatures above the polymerization temperature, after-treatment of the polymer solution by means of water vapor or stripping with nitrogen or treating the reaction mixture with oxidizing or reducing reagents, adsorption processes such as the adsorption of contamination onto selected media such as, for example, activated carbon, or ultrafiltration. The known work-up steps can also follow, for example suitable drying processes such as spray-drying, freeze-drying or drum-drying, or agglomeration processes following drying. The mixtures with a low residual monomer content obtained by the process according to the invention can also be sold directly.

Pulverulent polymers have the disadvantage of better storage properties, easier transportation and generally have a lower tendency for microbial attack.

### Neutralization

The polymers A according to the invention can be partially or completely neutralized. Particularly for using the polymers in hair cosmetic preparations, partial or complete neutralization is advantageous. In preferred embodiments, the polymers are neutralized, for example, to at least 10%, preferably to at least 30%, further preferably to at least 40%, particularly preferably to at least 50%, very particularly preferably to at least 70% and in particular to at least 95%.

In a very particularly preferred embodiment, the polymers are neutralized to at least 99%. The neutralization is most preferably to at least 100%.

The neutralization can take place during or after the polymerization.

It is also advantageous if the neutralizing agent is added in a more than equivalent amount, equivalent amount being understood as meaning the amount which is at least required in order to neutralize all of the neutralizable groups of the polymers.

The neutralization can be carried out, for example, with
a mono-, di- or trialkanolamine having 2 to 5 carbon atoms in the alkanol radical, which is present, if appropriate, in etherified form, for example mono-, di- and triethanolamine, mono-, di and tri-n-propanolamine, mono-, di- and triisopropanolamine, 2-amino-2-methylpropanol and di(2-methoxyethyl)amine,
an alkanediolamine having 2 to 5 carbon atoms, for example 2-amino-2-methyl-propane-1,3-diol and 2-amino-2-ethylpropane-1,3-diol, or
a primary, secondary or tertiary alkylamine having in total 5 to 10 carbon atoms, for example N,N-diethylpropylamine or 3-diethylamino-1-propylamine.

Suitable alkali metal hydroxides for the neutralization are primarily sodium hydroxide, or potassium hydroxide and ammonium hydroxide.

Good neutralization results are often obtained with 2-amino-2-methylpropanol, triiso-propanolamine, 2-amino-2-ethylpropane-1,3-diol, N,N-dimethylaminoethanol or 3-diethylamino-1-propylamine.

In a preferred embodiment of the invention, amines comprising hydroxy groups from the group consisting of N,N-dimethylethanolamine, N-methyldiethanolamine, triethanolamine, 2-amino-2-methylpropanol and mixtures thereof are chosen for the neutralization.

In this connection, alkanolamines carrying secondary or tertiary amino groups can exhibit advantageous effects.

For neutralizing the polymers in the preparations and compositions according to the invention, silicone polymers comprising amino groups in particular are suitable. Suitable silicone polymers comprising amino groups are, for example, the silicone-aminopolyalkylene oxide block copolymers of WO 97/32917, the products Silsoft®A-843 (dimethicone bisamino hydroxypropyl copolyol) and Silsoft®A-858 (trimethylsilyl amodimethicone copolymer) (both Witco). In addition, the neutralization polymers of EP-A 1 035 144 and in particular the silicone-containing neutralization polymers according to claim 12 of EP-A 1 035 144 are also suitable.

To increase the storage stability of the polymer solutions, it is advantageous to partially neutralize the polymers following the preparation. A neutralization immediately after the polymerization within the range from 10 to 20 mol %, based on the total amount of acid groups, is particularly advantageous.

### Cosmetic Preparations

The polymers A described above are exceptionally suitable for producing cosmetic, in particular hair cosmetic, preparations. They serve here, for example, as polymeric film formers. They can be used and formulated universally into a very wide variety of cosmetic, preferably hair cosmetic, preparations and are compatible with the customary components.

The polymers A are advantageously suitable for producing elastic hairstyles coupled with strong setting (even at high atmospheric humidity). The polymers A according to the invention are characterized by good propellant gas compatibility, good solubility in aqueous/alcoholic solvent mixtures, in particular by suitability for use as optically clear low-VOC formulations and by good ability to be washed out and ability to be combed out without flaking effect. In addition, they improve hair treated with them in its sensorally perceptible properties, such as feel, volume, handlability, etc. Hairspray formulations based on the polymers A according to the invention are characterized by good sprayability and good rheological properties and exceptionally low stickiness of the resulting films. The cosmetic, preferably hair cosmetic, preparations according to the invention comprising the polymers A do not have a tendency to form foam following application. Besides the good compatibility with the customary cosmetic ingredients, the applied polymers A dry quickly.

### Cosmetically Acceptable Carrier B)

The cosmetic preparations according to the invention are preferably aqueous preparations which comprise at least 10% by weight, preferably at least 20% by weight and particularly preferably at least 30% by weight of water. Besides water and the polymers A, the cosmetic preparations according to the invention also have at least one cosmetically acceptable carrier B) which is chosen from
i) water-miscible organic solvents, preferably C₂-C₄-alkanols, in particular ethanol,
ii) oils, fats, waxes,
iii) esters of C₆-C₃₀-monocarboxylic acids with mono-, di- or trihydric alcohols which are different from iii),
iv) saturated acyclic and cyclic hydrocarbons,
v) fatty acids,
(vi) fatty alcohols,
vii) propellants (propellant gases) and mixtures thereof.

Suitable carriers B and further active ingredients and additives to be used advantageously are described in detail below.

Suitable cosmetically and pharmaceutically compatible oil and fat components B) are described in Karl-Heinz Schrader, Grundlagen und Rezepturen der Kosmetika, [Fundamentals and formulations of cosmetics], 2nd Edition, Verlag Hüthig, Heidelberg, pp. 319-355, which is hereby incorporated by reference. The preparations according to the invention can, for example, have an oil or fat component B) which is chosen from: hydrocarbons of low polarity, such as mineral oils; linear saturated hydrocarbons, preferably having more than 8 carbon atoms, such as tetradecane, hexadecane, octadecane, etc.; cyclic hydrocarbons, such as decahydronaphthalene; branched hydrocarbon; animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate, benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name cyclomethicone.

Preferred oil and fat components B) are chosen from paraffin and paraffin oils; vaseline; natural fats and oils, such as castor oil, soybean oil, peanut oil, olive oil, sunflower oil, sesame oil, avocado oil, cocoa butter, almond oil, persic oil, ricinus oil, cod liver oil, lard, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candelilla wax, spermaceti and mixtures of the abovementioned oil and fat components.

Suitable hydrophilic carriers B) are chosen from water, 1-, 2- or polyhydric alcohols having preferably 1 to 8 carbon atoms, such as ethanol, n-propanol, isopropanol, propylene glycol, glycerol, sorbitol, etc.

The cosmetic preparations according to the invention may be skin cosmetic, hair cosmetic, dermatological, hygiene or pharmaceutical compositions. On account of their film-forming and flexible properties, the polymers A described above are particularly suitable as additives for hair and skin cosmetics.

Preferably, the preparations according to the invention which comprise the polymers A are in the form of a spray, gel, foam, ointment, cream, emulsion, suspension, lotion, milk or paste. If desired, liposomes or microspheres can also be used.

Preferably, the cosmetic compositions according to the invention comprise at least one polymer A as defined above, at least one carrier B as defined above and at least one constituent different therefrom which is preferably chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tints, tanning agents, dyes, pigments, consistency regulators, humectants, refatting agents, collagen, protein hydrolysates, lipids, antioxidants, antifoams, antistats, emollients and softeners.

The preparations according to the invention preferably have a pH of from 2.0 to 9.3. The pH range is particularly preferably between 4 and 8. Organic solvents or a mixture of solvents with a boiling point below 400° C. may be present as additional cosolvents in an amount of from 0.1 to 15% by weight, preferably from 1 to 10% by weight. Particularly suitable additional cosolvents are unbranched or branched hydrocarbons, such as pentane, hexane, isopentane and cyclic hydrocarbons, such as cyclopentane and cyclohexane. Further particularly preferred water-soluble solvents are glycerol, ethylene glycol and propylene glycol in an amount up to 30% by weight.

In a preferred embodiment of the invention, the preparations according to the invention have a fraction of volatile organic components of at most 80% by weight, preferably at most 55% by weight and in particular at most 35% by weight. A preferred subject-matter are thus cosmetic, preferably hair cosmetic, preparations which correspond to the low-VOC standard, i.e. VOC-80 or VOC-55 standard.

Preference is given to the use of the polymers A in particular in hairspray preparations which comprise the following constituents:
partially or completely neutralized polymer A according to the invention;
water;
cosmetically customary organic solvent such as, for example, ethanol, isopropanol and dimethoxymethane, in addition also acetone, n-propanol, n-butanol, 2-methoxypropan-1-ol, n-pentane, n-hexane, cyclohexane, n-heptane, n-octane or dichloromethane or mixtures thereof;
cosmetically customary propellant such as, for example, n-propane, isopropane, n-butane, isobutane, 2,2-dimethylbutane, n-pentane, isopentane, dimethyl ether, difluoroethane, fluorotrichloromethane, dichlorodifluoromethane or dichlorotetrafluoroethane, HFC-152 A (1,1-difluoroethane), HFC-134a (1,1,2,2-tetrafluoroethane), N₂, N₂O and CO or mixtures thereof.

To neutralize the polymers A according to the invention and thus component B) and to adjust the pH of the cosmetic, preferably hair cosmetic, preparations, alkanolamines are advantageously used. Examples (INCI) are aminomethylpropanol, diethanolamine, diisopropanolamine, ethanolamine, methylethanolamine, N-lauryldiethanolamine, triethanolamine, triisopropanolamine, etc. It is possible to use alkanolamines carrying either primary amino groups or secondary amino groups.

Furthermore, alkali metal hydroxides (e.g. NaOH, preferably KOH) and other bases can be used for the neutralization (e.g. histidine, arginine, lysine or ethylenediamines, diethylenetriamine, melamine, benzoguanamine). All of the bases given can be used on their own or as a mixture with other bases for the neutralization of acid-containing cosmetic products.

Accordingly, the present invention provides aqueous cosmetic, preferably hair cosmetic, preparations which, besides the at least one polymer A and the carrier B, also comprises at least one active ingredient or additive chosen from the group consisting of viscosity-modifying substances, haircare substances, hair-setting substances, silicone compounds, photoprotective substances, fats, oils, waxes, preservatives, pigments, soluble dyes, particulate substances, and surfactants.

In a preferred embodiment, hair cosmetic formulations according to the invention comprise

i) 0.05 to 20% by weight of at least one polymer A,
ii) 20 to 99.95% by weight of water and/or alcohol,
iii) 0 to 50% by weight of at least one propellant,
iv) 0 to 5% by weight of at least one emulsifier,
v) 0 to 3% by weight of at least one thickener, and
vi) up to 25% by weight of further constituents.

Alcohol is understood as meaning all of the abovementioned alcohols customary in cosmetics, e.g. ethanol, isopropanol, n-propanol.

### Propellants (Propellant Gases)

Of the specified compounds, the propellants (propellant gases) used are primarily the hydrocarbons, in particular propane, n-butane, n-pentane and mixtures thereof, and also dimethyl ether and difluoroethane. If appropriate, one or more of the specified chlorinated hydrocarbons are co-used in propellant mixtures, but only in small amounts, for example up to 20% by weight, based on the propellant mixture.

The cosmetic, preferably hair cosmetic, preparations according to the invention are also particularly suitable for pump spray preparations without the addition of propellants or else for aerosol sprays with customary compressed gases, such as nitrogen, compressed air or carbon dioxide as propellant.

A hydrous standard aerosol spray formulation comprises, for example, the following constituents:
a polymer according to the invention neutralized to 100% with 2-amino-2-methylpropanol
alcohol
water
dimethyl ether and/or propane/n-butane and/or propane/isobutane.
Here, the total amount of volatile organic components is preferably at most 80% by weight, particularly preferably at most 55% by weight, of the preparation.
Preferably, the cosmetic, preferably hair cosmetic, preparations according to the invention comprise at least one polymer A according to the invention, at least one cosmetically acceptable carrier B) as defined above and at least one further active ingredient or additive different therefrom which is chosen from cosmetically active ingredients, emulsifiers, surfactants, preservatives, perfume oils, thickeners, hair polymers, hair conditioners, graft polymers, water-soluble or dispersible silicone-containing polymers, photoprotective agents, bleaches, gel formers, care agents, colorants, tints, tanning agents, dyes, pigments, consistency regulators, humectants, refatting agents, collagen, protein hydrolysates, lipids, antioxidants, antifoams, antistats, emollients, lanolin components, protein hydrolysates and softeners.

### Further Polymers

To adjust the properties of cosmetic, preferably hair cosmetic, preparations in a targeted manner, it may be advantageous to use the polymers according to the invention in mixture with further (hair) cosmetically customary polymers.

In a further preferred embodiment, the composition according to the invention comprises 0.01 to 15% by weight, preferably 0.5 to 10% by weight, of at least one synthetic or natural nonionic, preferably a film-forming polymer. Natural polymers are also understood as meaning chemically modified polymers of natural origin. Film-forming polymers are understood as meaning those polymers which, when applied in 0.01 to 5% strength aqueous, alcoholic or aqueous-alcoholic solution, are able to deposit a polymer film on the hair.

Suitable such further customary polymers for this are, for example, anionic, cationic, amphoteric, zwitterionic and neutral polymers.

Examples of such further polymers are
copolymers of ethyl acrylate and methacrylic acid
copolymers of N-tert-butylacrylamide, ethyl acrylate and acrylic acid,
polyvinylpyrrolidones
polyvinylcaprolactams
polyurethanes
copolymers of acrylic acid, methyl methacrylate, octylacrylamide, butylaminoethyl methylacrylate and hydroxypropyl methacrylate,
copolymers of vinyl acetate and crotonic acid and/or (vinyl) neodecanoate,
copolymers of vinyl acetate and/or vinyl propionate and N-vinylpyrrolidone,
carboxy-functional copolymers of vinylpyrrolidone, t-butyl acrylate, methacrylic acid,
copolymers of tert-butyl acrylate, methacrylic acid and dimethicone copolyol.

Surprisingly, it has been found that cosmetic and preferably hair cosmetic preparations which comprise the polymers A in combination with further polymers have unexpected properties. The cosmetic and preferably hair cosmetic preparations according to the invention are superior to the preparations from the prior art especially with regard to the totality of their cosmetic properties.

Copolymers of ethyl acrylate and methacrylic acid (INCI name: Acrylates Copolymer) are available, for example, as commercial products Luviflex®Soft (BASF).

Copolymers of N-tert-butylacrylamide, ethyl acrylate and acrylic acid (INCI name: acrylates/acrylamide copolymer) are available, for example, as commercial products Ultrahold Strong®, Ultrahold 8® (BASF).

Polyvinylpyrrolidones (INCI name: PVP) are available, for example, under the trade names Luviskol®K, Luviskol®K30 (BASF) and PVP K® (ISP).

Polyvinylcaprolactams (INCI: polyvinylcaprolactams) are available, for example, under the trade name Luviskol Plus® (BASF).

Polyurethanes (INCI: Polyurethane-1) are available, for example, under the trade name Luviset®PUR.

Copolymers of acrylic acid, methyl methacrylate, octylacrylamide, butylaminoethyl methylacrylate, hydroxypropyl methacrylate (INCI: Octylacrylamide/Acrylates/Butyl-aminoethyl Methacrylate Copolymer) are known, for example, under the trade names Amphomer®28-4910 and Amphomer®LV-71 (National Starch).

Copolymers of vinyl acetate and crotonic acid (INCI: VA/Crotonate/Copolymer) are available, for example, under the trade names Luviset®CA 66 (BASF), Resyn®28-1310 (National Starch), Gafset® (GAF) or Aristoflex®A (Celanese).

Copolymers of vinyl acetate, crotonic acid and (vinyl)neodecanoate (INCI: VA/Crotonates/Neodecanoate Copolymer) are available, for example, under the trade names Resyn®28-2930 (National Starch) and Luviset®CAN (BASF).

Copolymers of vinyl acetate and N-vinylpyrrolidone (INCI: PVP/VA) are available, for example, under the trade names Luviskol VA® (BASF) and PVPIVA (ISP).

Carboxyfunctional copolymers of vinylpyrrolidone, t-butyl acrylate, methacrylic acid are available, for example, under the trade name Luviskol®VBM (BASF).

Copolymers of tert-butyl acrylate, methacrylic acid and dimethicone copolyol are available, for example, under the trade name Luviflex®Silk (BASF).

Suitable anionic polymers are homopolymers and copolymers of acrylic acid and methacrylic acid or salts thereof which are different from the polymers A, copolymers of acrylic acid and acrylamide and salts thereof, sodium salts of polyhydroxycarboxylic acids, copolymers of acrylic acid and methacrylic acid with, for example, hydrophobic monomers, e.g. C₄-C₃₀-alkyl esters of (meth)acrylic acid, C₄-C₃₀-alkylvinyl esters, C₄-C₃₀-alkyl vinyl ethers and hyaluronic acid and also further polymers known under the trade names Amerhold®DR-25, Ultrahold®, Luviset®P.U.R., Acronal®, Acudyne®, Lovocryl®, Versatyl®, Amphomer® (28-4910, LV-71), Placise®L53, Gantrez®ES 425, Advantage Plus®, Omnirez®2000, Resyn®28-1310, Resyn®28-2930, Balance®(0/55), Acudyne®255, Aristoflex®A or Eastman AQ®.

In addition, the group of suitable polymers comprises, for example, Balance®CR (National Starch), Balance®47 (National Starch; octylacrylamide/-acrylates/butylaminoethyl methacrylates copolymer), Aquaflex®FX 64 (ISP; isobutylene/ethylmaleimide/hydroxyethylmaleimide copolymer), Aquaflex®SF-40 (ISP/National Starch; VP/vinyl caprolactam/DMAPA acrylates copolymer), Allianz®LT-120 (ISP/Rohm & Haas; acrylate/C1-2 succinate/hydroxyacrylate copolymer), Aquarez® HS (Eastman; Polyester-1).

Also suitable are the polymers under the trade names Diaformer®Z-400 (Clariant; methacryloylethylbetaine/methacrylate copolymer), Diaformer®Z-711 (Clariant; methacryloylethyl N-oxide/methacrylate copolymer), Diaformer®Z-712 (Clariant; methacryloylethyl N-oxide/methacrylate copolymer), Omnirez®2000 (ISP; monoethyl ester of poly(methyl vinyl ether/maleic acid in ethanol), Amphomer®HC (National Starch; acrylate/octylacrylamide copolymer), Amphomer®28-4910 (National Starch; octylacrylamide/acrylate/butylaminoethyl methcrylate copolymer), Advantage®HC 37 (ISP; terpolymer of vinylcaprolactam/vinylpyrrolidone/dimethylaminoethyl methacrylate), Advantage®LC55 and LC80 or LC A and LC E, Advantage®Plus (ISP; VA/butyl maleate/isobornyl acrylate copolymer), Aculyne®258 (Rohm & Haas; acrylate/hydroxy ester acrylate copolymer), Luviset®P.U.R. (BASF, Polyurethane-1), Eastman®AQ 48 (Eastman), Styleze®CC-10 (ISP; VP/DMAPA acrylates copolymer), Styleze® 2000 (ISP; VP/acrylates/laurylmethacrylate copolymer), DynamX® (National Starch; polyurethane-14 AMP acrylates copolymer), Resyn®XP (National Starch; acrylates/octylacrylamide copolymer), Fixomer® A-30 (Ondeo Nalco; polymethacrylic acid (and) acrylamidomethylpropanesulfonic acid), Fixate® G-100 (Noveon; AMP acrylates/allyl methacrylate copolymer).

Suitable polymers are also copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® (Rohm+Haas). Particularly suitable polymers are also copolymers of t-butyl acrylate, ethyl acrylate, methacrylic acid (e.g. Luvimer®100P, Luvimer®Pro55) and copolymers of ethyl acrylate and methacrylic acid (e.g. Luvimer®MAE).

Also suitable are crosslinked polymers of acrylic acid, as are available under the INCI name Carbomer. Such crosslinked homopolymers of acrylic acid are commercially available, for example, as Carbopol® (Noveon). Preference is also given to hydrophobically modified crosslinked polyacrylate polymers, such as Carbopol®Ultrez 21 (Noveon). Such further polymers can also be used for modifying the rheology of the preparations, i.e. as thickeners.

Further suitable additional polymers are water-soluble or water-dispersible polyesters, polyureas, polyurethanes, polyurethaneureas, maleic anhydride copolymers reacted, if appropriate, with alcohols, or anionic polysiloxanes.

In addition, polymers suitable for use together with the polymers A are, for example, also cationic and cationogenic polymers. These include, for example,
copolymers of N-vinylpyrrolidone/N-vinylimidazolium salts (available, for example, under the trade names Luviquat®FC, Luviquat®HM, Luviquat®MS, Luviquat®Care, Luviquat® UltraCare (BASF),
copolymers of vinylpyrrolidone, methacrylamide, vinyl imidazole (Luviset®Clear)
copolymers of N-vinylcaprolactam/N-vinylpyrrolidone/N-vinylimidazolium salts (available, for example, under the trade name Luviquat® Hold),
copolymers of N-vinylpyrrolidone/dimethylaminoethyl methacrylate, quaternized with diethyl sulfate (available, for example, under the trade name Luviquat®PQ11),
cationic cellulose derivatives (polyquaternium-4 and -10),
acrylamide copolymers (polyquaternium-7),
guar hydroxypropyltrimethylammonium chloride (INCI: Hydroxypropyl Guar Hydroxypropyltrimonium Chloride),
polyethyleneimines and salts thereof,
polyvinylamines and salts thereof,
polymers based on dimethyldiallylammonium chloride (Merquat®),
polymers which are formed by reacting polyvinylpyrrolidone with quaternary ammonium compounds (Gafquat®),
hydroxyethylcellulose with cationic groups (Polymer®JR) and
cationic plant-based polymers, e.g. guar polymers, such as the Jaguar® grades from Rhodia.

Suitable as further hair cosmetic polymers are also neutral polymers, such as
polyvinylpyrrolidones,
copolymers of N-vinylpyrrolidone and vinyl acetate and/or vinyl propionate,
polysiloxanes,
polyvinylcaprolactams and
copolymers with N-vinylpyrrolidone,
cellulose derivatives,
polyaspartic acid salts and derivatives,
polyamides, e.g. based on itaconic acid and aliphatic diamines, as described in DE-A-43 33 238.

The abovementioned types of polymer include those known under the trade names Luviskol® (K, VA, Plus), PVP K, PVP/VA, Advantage®HC, Luviflex®Swing, Kollicoat®IR, H₂OLD®EP-1.

Furthermore, suitable further polymers are also biopolymers, i.e. polymers which are obtained from naturally renewable raw materials and are constructed from natural monomer building blocks, e.g. cellulose derivatives, chitin, chitosan, DNA, hyaluronic acid and RNA derivatives.

Suitable mixing partners for the polymers according to the invention are also zwitterionic polymers, as are disclosed, for example, in the German patent applications DE 39 29 973, DE 21 50 557, DE 28 17 369 and DE 37 08 451, and methacroylethylbetaine/methacrylate copolymers which are commercially available under the name Amersette® (Amerchol), or copolymers of hydroxyethyl methacrylate, methyl methacrylate, N,N-dimethylaminoethyl methacrylate and acrylic acid (Jordapon®).

Further suitable polymers are also betainic polymers, such as Yukaformers (R205, SM) and Diaformers.

Polymers suitable as mixing partners are also nonionic, siloxane-containing, water-soluble or -dispersible polymers, e.g. polyether siloxanes, such as Tegopren® (Goldschmidt) or Belsil® (Wacker).

Cosmetically and/or Dermatologically Active Ingredients

Suitable cosmetically and/or dermatologically active ingredients are, for example, coloring active ingredients, skin and hair pigmentation agents, tints, tanning agents, bleaches, keratin-hardening substances, antimicrobial active ingredients, photo filter active ingredients, repellant active ingredients, hyperemic substances, keratolytic and keratoplastic substances, antidandruff active ingredients, antiphlogistics, keratinizing substances, antioxidative active ingredients or active ingredients which act as free-radical scavengers, substances which moisten the skin or keep the skin moist, refatting active ingredients, antierythimatous or antiallergic active ingredients and mixtures thereof.

Preferred cosmetic care and active ingredients are AHA acids, fruit acids, ceramides, phytantriol, collagen, vitamins and provitamins, for example vitamin A, E and C, retinol, bisabolol and panthenol. A particularly preferred cosmetic care substance in the preparations according to the invention is panthenol, which is commercially available, for example, as D-Panthenol®USP, D-Panthenol®50 P, D-Panthenol®75 W, D,L-Panthenol®50 W.

Artificially skin-tanning active ingredients which are suitable for tanning the skin without natural or artificial irradiation with UV rays are, for example, dihydroxyacetone, alloxan and walnut shell extract.

Suitable keratin-hardening substances are generally active ingredients as are also used in antiperspirants, such as, for example, potassium aluminum sulfate, aluminum hydroxychloride, aluminum lactate, etc.

Antimicrobial active ingredients are used to destroy microorganisms or to inhibit their growth and thus serve both as preservatives and also as deodorizing substance which reduces the formation or the intensity of body odor. These include, for example, customary preservatives known to the person skilled in the art, such as p-hydroxy-benzoic esters, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine, etc. The preparations according to the invention comprise preferably 0.01 to 5% by weight, particularly preferably 0.05 to 1% by weight, of at least one preservative. Suitable further preservatives are the substances listed in the International Cosmetic Ingredient Dictionary and Handbook, 9th Edition with the function “Preservatives”, e.g. phenoxyethanol, benzyl paraben, butyl paraben, ethyl paraben, isobutyl paraben, isopropyl paraben, methyl paraben, propyl paraben, iodopropynyl butylcarbamate, methyldibromoglutaronitrile, DMDM hydantoin.

### UV Filter Substances

In one embodiment, the preparations according to the invention can comprise oil-soluble and/or water-soluble UVA and/or UVB filters.

The total amount of the filter substances is preferably 0.01 to 10% by weight or from 0.1 to 5% by weight, particularly preferably from 0.2 to 2% by weight, based on the total weight of the preparations.

The majority of the photoprotective agents in the preparations serving to protect the human epidermis consists of compounds which absorb UV light in the UV-B region. For example, the fraction of UV-A absorbers to be used according to the invention is 10 to 90% by weight, preferably 20 to 50% by weight, based on the total amount of UV-B and UV-A absorbing substances.

The UVB filters may be oil-soluble or water-soluble. Advantageous UVB filter substances are, for example:
i) benzimidazolsulfonic acid derivatives, such as, for example, 2-phenylbenzimidazol-5-sulfonic acid and salts thereof
ii) benzotriazole derivatives, such as, for example, 2,2′-methylenebis(6-(2H-benzotriazol-2-yl)-4-(1,1,3,3-tetramethylbutyl)phenol)
iii) 4-aminobenzoic acid derivatives, preferably 2-ethylhexyl 4-(dimethylamino)benzoate, amyl 4-(dimethylamino)benzoate;
iv) esters of benzalmalonic acid, preferably di(2-ethylhexyl) 4-methoxybenzalmalonate;
v) esters of cinnamic acid, preferably 2-ethylhexyl 4-methoxycinnamate, isopentyl 4-methoxycinnamate;
vi) derivatives of benzophenone, preferably 2-hydroxy-4-methoxybenzophenone, 2-hydroxy-4-methoxy-4′-methylbenzophenone, 2,2′-dihydroxy-4-methoxybenzophenone;
vii) methylidenecamphor derivatives, preferably 4-methylbenzylidenecamphor, benzylidenecamphor;
viii) triazine derivatives, preferably tris(2-ethylhexyl) 4,4′,4″-(1,3,5-triazine-2,4,6-triylimino)trisbenzoate [INCI: Diethylhexyl Butamido Triazine, UVA-Sorb® HEB (Sigma 3V)] and 2,4,6-tris[anilino(p-carbo-2′-ethyl-1′-hexyloxy)]-1,3,5-triazine [INCI: Octyl Triazone, Uvinul®T 150 (BASF)].

Water-soluble UVB filter substances to be used advantageously are, for example, sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

UVA filters to be used advantageously are, for example:
1,4-phenylenedimethinecamphorsulfonic acid derivatives, such as, for example, 3,3′-(1,4-phenylenedimethine)bis(7,7-dimethyl-2-oxobicyclo[2.2.1]heptane-1-methanesulfonic acid and its salts
1,3,5-triazine derivatives, such as 2,4-bis{[(2-ethylhexyloxy)-2-hydroxy)phenyl}-6-(4-methoxyphenyl)-I,3,5)-triazine (e.g. Tinosorb®S (Ciba))
dibenzoylmethane derivatives, preferably 4-isopropyldibenzoylmethane, 4-(tert-butyl)-4′-methoxydibenzoylmethane
benzoxazole derivatives, for example 2,4-bis[4-[5-(1,1-dimethylpropyl)benzoxazol-2-yl]phenylimino]-6-[(2-ethylexyl)imino]-1,3,5-triazine (CAS No. 288254-16-0, Uvasorb®K2A (3V Sigma))
hydroxybenzophenones, for example hexyl 2-(4′-diethylamino-2′-hydroxybenzoyl)benzoate (also: aminobenzophenone) (Uvinul®A Plus (BASF))

In addition, it may, if appropriate, be advantageous according to the invention to provide preparations with further UVA and/or UVB filters, for example certain salicylic acid derivatives, such as 4-isopropylbenzyl salicylate, 2-ethylhexyl salicylate, octyl salicylate, homomethyl salicylate. The total amount of salicylic acid derivatives in the cosmetic preparations is advantageously chosen from the range from 0.1-15.0% by weight, preferably 0.3-10.0% by weight, based on the total weight of the preparations. A further photoprotective filter to be used advantageously according to the invention is ethylhexyl 2-cyano-3,3-diphenylacrylate (octocrylene, Uvinul®N 539 (BASF)).

The table below lists by way of example some of the photoprotective filters suitable for use in the preparations according to the invention:

| No. | Substance | CAS No. |
|---|---|---|
| 1 | 4-Aminobenzoic acid | 150-13-0 |
| 2 | 3-(4′-Trimethylammonium)benzylidenebornan-2-one methyl | 52793-97-2 |
| | sulfate | |
| 3 | 3,3,5-Trimethylcyclohexyl salicylate (homosalate) | 118-56-9 |
| 4 | 2-Hydroxy-4-methoxybenzophenone (oxybenzone) | 131-57-7 |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl-2- | 90457-82-2 |
| | oxobicyclo[2.2.1]heptane-1-methanesulfonic acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl)-2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (dioxybenzone) | 131-53-3 |
| 23 | 2-Hydroxy-4-methoxy-4-methylbenzophenone (mexenone) | 1641-17-4 |
| 24 | Triethanolamine salicylate | 2174-16-5 |
| 25 | Dimethoxyphenylglyoxalic acid or: | 4732-70-1 |
| | 3,4-dimethoxyphenylglyoxal acidic sodium | |
| 26 | 3-(4′Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-methylenebis[6-(2H-benzotriazol-2-yl)-4-(1,1,3,3,- | 103597-45-1 |
| | tetramethylbutyl)phenol] | |
| 30 | 2,2′-(1,4-phenylene)bis-1H-benzimidazole-4,6-disulfonic acid, Na | 180898-37-7 |
| | salt | |
| 31 | 2,4-bis[4-(2-Ethylhexyloxy)-2-hydroxy]phenyl- | 187393-00-6 |
| | 6-(4-methoxyphenyl)(1,3,5)-triazine | |
| 32 | 3-(4-methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone 5,5′- | 3121-60-6 |
| | disodium sulfonate | |
| 36 | Benzoic acid, 2-[4-(diethylamino)-2-hydroxybenzoyl]-, hexyl ester | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

Suitable UV photoprotective filters with the CAS No. 113010-52-9 are commercially available, for example, under the name Uvinul®P 25.

Polymeric or polymer-bound filter substances can also be used according to the invention.

Metal oxides such as titanium dioxide or zinc oxide can likewise be used advantageously for protecting against harmful solar radiation. Their effect is essentially based on reflection, scattering and absorption of the harmful UV radiation and essentially depends on the primary particle size of the metal oxides. The cosmetic preparations according to the invention can, furthermore, advantageously comprise inorganic pigments based on metal oxides and/or other metal compounds which are insoluble or sparingly soluble in water, chosen from the group of oxides of zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. They are particularly preferably pigments based on ZnO.

The inorganic pigments can here be present in coated form, i.e. that they are treated superficially. This surface treatment can, for example, consist in providing the pigments with a thin hydrophobic layer by a method known per se, as described in DE-A-33 14 742.

Photoprotective agents suitable for use in the preparations according to the invention are the compounds specified in EP-A 1 084 696 in paragraphs [0036] to [0053], which is hereby incorporated in its entirety at this point by reference. Of suitability for the use according to the invention are all UV photoprotective filters which are specified in Annex 7 (to § 3b) of the German Cosmetics Directive under “Ultraviolet filters for cosmetic compositions”.

The list of specified UV photoprotective filters which can be used in the preparations according to the invention is not exhaustive.

### Thickeners

Suitable thickeners are specified in “Kosmetik und Hlygiene von Kopf bis Fuβ” [Cosmetics and hygiene from head to foot], Ed. W. Umbach, 3rd Edition, Wiley-VCH, 2004, pp. 235-236, which is hereby incorporated in its entirety at this point by reference.

Consistency regulators allow the desired viscosity of, for example, shampoos to be set. Thickeners which have a viscosity-increasing effect due to the surfactant micelles increasing in size or due to swelling of the water phase originate from chemically very different classes of substances.

Suitable thickeners for the preparations according to the invention are crosslinked polyacrylic acids and derivatives thereof, polysaccharides such as xanthan gum, guar guar, agar agar, alginates or tyloses, cellulose derivatives, e.g. carboxymethylcellulose or hydroxycarboxymethylcellulose, also higher molecular weight polyethylene glycol mono- and diesters of fatty acids, fatty alcohols, monoglycerides and fatty acids, polyvinyl alcohol and polyvinylpyrrolidone.

Suitable thickeners are also polyacrylates, such as Carbopol® (Noveon), Ultrez® (Noveon), Luvigel® EM (BASF), Capigel®98 (Seppic), Synthalene® (Sigma), the Aculyn® grades from Rohm and Haas, such as Aculyn® 22 (copolymer of acrylates and methacrylic acid ethoxylates with stearyl radical (20 EO units)) and Aculyn® 28 (copolymer of acrylates and methacrylic acid ethoxylates with behenyl radical (25 EO units)).

Suitable thickeners are also, for example, Aerosil grades (hydrophilic silicas), polyacrylamides, polyvinyl alcohol and polyvinylpyrrolidone, surfactants such as, for example, ethoxylated fatty acid glycerides, esters of fatty acids with polyols, such as, for example, pentaerythritol or trimethylolpropane, fatty alcohol ethoxylates with a narrowed homolog distribution or alkyl oligoglucosides, and electrolytes, such as sodium chloride and ammonium chloride.

Particularly preferred thickeners for producing gels are Ultrez®21, Aculyn®28, Luvigel® EM and Capigel® 98.

Particularly in the case of more highly concentrated shampoo formulations it is also possible, to regulate the consistency, to add substances which reduce the viscosity of the formulation, such as, for example, propylene glycol and glycerol. These substances influence the product properties only slightly.

### Gel Formers

If the use of gel formers is desired for the preparations according to the invention, then all gel formers customary in cosmetics can be used. These include slightly crosslinked polyacrylic acid, for example Carbomer (INCI), cellulose derivatives, e.g. Hydroxypropylcellulose, Hydroxyethylcellulose, cationically modified celluloses, polysaccharides, e.g. Xanthan gum, Caprylic/Capric Triglyceride, Sodium Acrylates Copolymer, Polyquaternium-32 (and) Paraffinum Liquidum (INCI), Sodium Acrylates Copolymer (and) Paraffinum Liquidum (and) PPG-1 Trideceth-6, Acrylamidopropyl Trimonium Chloride/Acrylamide Copolymer, Steareth-10 Allyl Ether Acrylates Copolymer, Polyquaternium-37 (and) Paraffinum Liquidum (and) PPG-1 Trideceth-6, Polyquaternium 37 (and) Propylene Glycole Dicaprate Dicaprylate (and) PPG-1 Trideceth-6, Polyquaternium-7, Polyquaternium-44.

### Emulsifiers

Suitable emulsifiers are, for example, nonionogenic surfactants from at least one of the following groups:
Addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
C12/18 fatty acid mono- and diesters of addition products of from 1 to 30 mol of ethylene oxide onto glycerol;
glycerol mono- and diesters and sorbitan mono- and diesters of saturated and unsaturated fatty acids having 6 to 22 carbon atoms and ethylene oxide addition products thereof;
alkyl mono- and oligoglycosides having 8 to 22 carbon atoms in the alkyl radical and ethoxylated analogues thereof;
addition products of from 15 to 60 mol of ethylene oxide onto castor oil and/or hydrogenated castor oil;
polyol and in particular polyglycerol esters, such as, for example, polyglycerol polyricinoleate, polyglycerol poly-12-hydroxystearate or polyglycerol dimerate. Likewise suitable are mixtures of compounds from two or more of these classes of substance;
addition products of from 2 to 15 mol of ethylene oxide onto castor oil and/or hydrogenated castor oil;
partial esters based on linear, branched, unsaturated or saturated C_{6/22}-fatty acids, ricinoleic acid and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose);
mono-, di- and trialkyl phosphates, and mono-, di- and/or tri-PEG alkyl phosphates and salts thereof;
wool wax alcohols;
polysiloxane-polyalkyl-polyether copolymers and corresponding derivatives;
mixed esters of pentaerythritol, fatty acids, citric acid and fatty alcohol according to German patent 1165574 and/or mixed esters of fatty acids having 6 to 22 carbon atoms, methylglycose and polyols, preferably glycerol or polyglycerol and
polyalkylene glycols.

The addition products of ethylene oxide and/or of propylene oxide onto fatty alcohols, fatty acids, alkylphenols, glycerol mono- and diesters, and also sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. C₁₂ to C₁₈-fatty acid mono- and diesters of addition products of ethylene oxide onto glycerol are known from German patent 2024051 as refatting agents for cosmetic preparations. C₈ to C₁₈-alkyl mono- and oligoglycosides, their preparation and their use are known from the prior art. Their preparation takes place in particular by reacting glucose or oligosaccharides with primary alcohols having 8 to 18 carbon atoms. With regard to the glycoside ester, both monoglycosides in which a cyclic sugar radical is glycosidically bonded to the fatty alcohol, and also oligomeric glycosides with a degree of oligomerization up to preferably about 8 are suitable. The degree of oligomerization here is a statistical average value which is based on a homolog distribution customary for such technical-grade products.

In addition, zwitterionic surfactants can be used as emulsifiers. Zwitterionic surfactants is the term used to refer to those surface-active compounds which carry at least one quaternary ammonium group and at least one carboxylate group and/or one sulfonate group in the molecule. Particularly suitable zwitterionic surfactants are the so-called betaines, such as the N-alkyl-N,N-dimethylammonium glycinates, for example coco-alkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines each having 8 to 18 carbon atoms in the alkyl or acyl group, and cocoacylaminoethyl hydroxyethylcarboxymethyl glycinate.

Particular preference is given to the fatty acid amide derivative known under the CTFA name Cocamidopropyl Betaine. Likewise suitable emulsifiers are ampholytic surfactants. Ampholytic surfactants are understood as meaning those surface-active compounds which, apart from a C₈ to C₁₈-alkyl or -acyl group in the molecule, comprise at least one free amino group and at least one —COOH and/or —SO₃H group and are capable of forming internal salts. Examples of suitable ampholytic surfactants are N-alkylglycines, N-alkylpropionic acids, N-alkylaminobutyric acids, N-alkylimino-dipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂ to C₁₈-acylsarcosine.

Besides the ampholytic emulsifiers, quaternary emulsifiers are also suitable, particular preference being given to those of the esterquat type, preferably methyl-quaternized difatty acid triethanolamine ester salts.

### Antioxidants

An additional content of antioxidants in the preparations may be advantageous. According to the invention, antioxidants which may be used are all antioxidants which are customary or suitable for cosmetic applications. The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, γ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoxamine) in very low tolerated doses (e.g. pmol to μmol/kg), also (metal)chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, biutylhydroxytoluene, butylhydroxyanisol, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

The amount of the abovementioned antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to provide these in concentrations of from 0.001 to 10% by weight, based on the total weight of the preparation.

If vitamin A or vitamin A derivatives, or carotenes or derivatives thereof are the antioxidant or the antioxidants, it is advantageous to provide these in concentrations of from 0.001 to 10% by weight, based on the total weight of the preparation.

### Perfume Oils

The cosmetic, preferably hair cosmetic preparations can comprise perfume oils. Perfume oils which may be mentioned are, for example, mixtures of natural and synthetic fragrances. Natural fragrances are extracts of flowers (lily, lavendar, rose, jasmine, neroli, yiang-ylang), stems and leaves (geranium, patchouli, petitgrain), fruits (anise, coriander, caraway, juniper), fruit peels (bergamot, lemon, orange), roots (mace, angelica, celery, cardamom, costus, iris, calmus), woods (pine wood, sandalwood, guaiac wood, cedar wood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, 4-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonat, the ketones include, for example, the ionones, cc-isomethylionone and methyl cedryl ketone, the alcohols include anethof, citronellol, eugenol, isoeugenol, geraniol, linalool, phenylethyl alcohol and terioneol, and the hydrocarbons include primarily the terpenes and balsams. However, preference is given to using mixtures of different fragrances which together produce a pleasing scent note. Essential oils of lower volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, a-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, vertofix coeur, iso-E-super, Fixolide NP, evernyl, iraldein gamma, phenylacetic acid, geranyl acetate, benzyl acetate, rose oxide, rommilat, irotyl and floramat alone or in mixtures.

### Superfatting Agents

Superfatting agents which may be used are substances such as, for example, lanolin and lecithin, and polyethoxylated or acylated lanolin and lecithin derivatives, polyol fatty acid esters, monoglycerides and fatty acid alkanolamides, the latter serving as foam stabilizers at the same time.

### Silicone Compounds

In one embodiment, the preparations according to the invention comprise, as haircare additive, at least one silicone compound in an amount of preferably 0.01 to 15% by weight, particularly preferably from 0.1 to 5% by weight. The silicone compounds comprise volatile and nonvolatile silicones and silicones which are insoluble or soluble in the composition. In one embodiment, they are high molecular weight silicones with a viscosity of from 1000 to 2 000 000 cSt at 25° C., preferably 10 000 to 1 800 000 or 100 000 to 1 500 000. The silicone compounds comprise polyalkyl- and polyaryl-siloxanes, in particular with methyl, ethyl, propyl, phenyl, methylphenyl and phenyl-methyl groups. Preference is given to polydimethylsiloxanes, polydiethylsiloxanes, polymethylphenylsiloxane. Preference is also given to shine-imparting, arylated silicones with a refractive index of at least 1.46, or at least 1.52. The silicone compounds comprise in particular the substances with the INCI names cyclomethicone, dimethicone, dimethiconol, dimethicone copolyol, phenyl trimethicone, amodimethicone, trimethylsilylamodimethicone, stearyl siloxysilicate, polymethyl-silsesquioxane, dimethicone crosspolymer. Also suitable are silicone resins and silicone elastomers, which are highly crosslinked siloxanes.

Preferred silicones are cyclic dimethylsiloxanes, linear polydimethylsiloxanes, block polymers of polydimethylsiloxane and polyethylene oxide and/or polypropylene oxide, polydimethylsiloxanes with terminal or lateral polyethylene oxide or polypropylene oxide radicals, polydimethylsiloxanes with terminal hydroxyl groups, phenyl-substituted polydimethylsiloxanes, silicone emulsions, silicone elastomers, silicone waxes, silicone gums and amino-substituted silicones (CTFA: amodimethicones).

### Hair Conditioners

In one embodiment, the preparations according to the invention comprise 0.01 to 20% by weight, preferably from 0.05 to 10% by weight, particularly preferably from 0.1 to 5% by weight, of at least one conditioner.

Conditioning agents preferred according to the invention are, for example, all compounds which are listed in the International Cosmetic Ingredient Dictionary and Handbook (Volume 4, Editor: R. C. Pepe, J. A. Wenninger, G. N. McEwen, The Cosmetic, Toiletry, and Fragrance Association, 9th Edition, 2002) under Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and all of the compounds listed in EP-A 934 956 (pp. 11-13) under “water soluble conditioning agent” and “oil soluble conditioning agent”. Further advantageous conditioning agents are, for example, the compounds referred to in accordance with INCI as polyquaternium (in particular Polyquaternium-1 to Polyquaternium-56). Suitable conditioning agents include, for example, also polymeric quaternary ammonium compounds, cationic cellulose derivatives, chitosan derivatives and polysaccharides.

The conditioning agent is preferably chosen from betaine, panthenol, panthenyl ethyl ether, sorbitol, protein hydrolysates, plant extracts; A-B block copolymers of alkyl acrylates and alkyl methacrylates; A-B block copolymers of alkylmethacrylates and acrylonitrile; A-B-A block copolymers of lactide and ethylene oxide; A-B-A block copolymers of caprolactone and ethylene oxide; A-B-C block copolymers of alkylene or alkadiene compounds, styrene and alkyl methacrylates; A-B-C block copolymers of acrylic acid, styrene and alkyl methacrylates, star-shaped block copolymers, hyperbranched polymers, dendrimers, intrinsically electrically conductive 3,4-polyethylenedioxythiophenes and intrinsically electrically conductive polyanilines. Further conditioning agents advantageous according to the invention are cellulose derivatives and quaternized guar gum derivatives, in particular guar hydroxypropyl-ammonium chloride (e.g. Jaguar Excel®, Jaguar C 162® (Rhodia), CAS 65497-29-2, CAS 39421-75-5).

Nonionic poly-N-vinylpyrrolidone/polyvinyl acetate copolymers (e.g. Luviskol®VA 64 (BASF)), anionic acrylate copolymers (e.g. Luviflex®Soft (BASF)), and/or amphoteric amide/acrylate/methacrylate copolymers (e.g. Amphomer® (National Starch)) can also be used advantageously according to the invention as conditioning agents.

### Hydrotropes

To improve the flow behavior, hydrotropes, such as, for example, ethanol, isopropyl alcohol, or polyols, can also be used. Polyols which are suitable here have preferably 2 to 15 carbon atoms and at least two hydroxyl groups. Typical examples are
glycerol;
alkylene glycols, such as, for example, ethylene glycol, diethylene glycol, propylene glycol, butylene glycol, hexylene glycol, and polyethylene glycols with an average molecular weight of from 100 to 1000 daltons;
technical-grade oligoglycerol mixtures with a degree of self-condensation of from 1.5 to 10, such as, for example, technical-grade diglycerol mixtures with a diglycerol content of from 40 to 50% by weight;
methylol compounds, such as, in particular, trimethylolethane, trimethylolpropane, trimethylolbutane, pentaerythritol and dipentaerythritol;
lower alkyl glucosides, in particular those with 1 to 8 carbon atoms in the alkyl radical, such as, for example, methyl and butyl glucoside;
sugar alcohols having 5 to 12 carbon atoms, such as, for example, sorbitol or mannitol;
sugars having 5 to 12 carbon atoms, such as, for example, glucose or sucrose;
amino sugars, such as, for example, glucamine.

### Oils, Fats and Waxes

The cosmetic, preferably hair cosmetic preparations according to the invention can also comprise oils, fats or waxes. These are advantageously chosen from the group of lecithins and fatty acid triglycerides, namely the triglycerol esters of saturated and/or unsaturated, branched and/or unbranched alkanecarboxylic acids with a chain length of from 8 to 24, in particular 12 to 18, carbon atoms. The fatty acid triglycerides can, for example, be chosen advantageously from the group of synthetic, semisynthetic and natural oils, such as, for example, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and the like. Further polar oil components can be chosen from the group of esters of saturated and/or unsaturated, branched and/or unbranched alkanecarboxylic acids with a chain length from 3 to 30 carbon atoms and saturated and/or unsaturated, branched and/or unbranched alcohols with a chain length of from 3 to 30 carbon atoms, and from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononalnoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, dicaptylyl carbonate (Cetiol CC) and cocoglycerides (Myritol 331), butylene glycol dicaprylate/dicaprate and dibutyl adipate, and synthetic, semisynthetic and natural mixtures of such esters, such as, for example, jojoba oil.

In addition, one or more oil components can be chosen advantageously from the group of branched and unbranched hydrocarbons and hydrocarbon waxes, silicone oils, dialkyl ethers, the group of saturated or unsaturated, branched or unbranched alcohols. Any mixtures of such oil and wax components are also to be used advantageously for the purposes of the present invention. It may also, if appropriate, be advantageous to use waxes, for example cetyl palmitate, as the sole lipid component of the oil phase. According to the invention, the oil component is advantageously chosen from the group consisting of 2-ethylhexyl isostearate, octyldodecanol, isotridecyl isononanoate, isoeicosane, 2-ethylhexyl cocoate, C12-15-alkylbenzoate, caprylic-capric triglyceride, dicaprylyl ether.

Mixtures of C12-15-alkyl benzoate and 2-ethylhexyl isostearate, mixtures of C12-15-alkyl benzoate and isotridecyl isononanoate, and mixtures of C12-C15-alkyl benzoate, 2-ethylhexyl isostearate and isotridecyl isononanoate are advantageous according to the invention.

According to the invention, particular preference is given to using fatty acid triglycerides, in particular soybean oil and/or almond oil, as oils with a polarity of from 5 to 50 mN/m.

In addition, the oil phase can advantageously be chosen from the group of Guerbet alcohols. These are liquid even at low temperatures and cause virtually no skin irritations. They can be used advantageously as fatting, superfatting and also refatting constituents in cosmetic compositions.

The use of Guerbet alcohols in cosmetics is known per se.

Guerbet alcohols preferred according to the invention are 2-butyloctanol (available commercially, for example, as Isofol®12 (Condea)) and 2-hexyldecanol (available commercially, for example, as Isofol®16 (Condea)).

According to the invention, mixtures of Guerbet alcohols according to the invention are also to be used advantageously, such as, for example, mixtures of 2-butyloctanol and 2-hexyldecanol (commercially available, for example, as Isofol®14 (Condea)).

Any mixtures of such oil and wax components are also to be used advantageously for the purposes of the present invention. Among the polyolefins, polydecenes are the preferred substances.

Fat and/or wax components to be used advantageously according to the invention can be chosen from the group of vegetable waxes, animal waxes, mineral waxes and petrochemical waxes. For example, candelilla wax, carnauba wax, japan wax, esparto grass wax, cork wax, guaruma wax, ricegerm oil wax, sugarcane wax, berry wax, ouricury wax, montan wax, jojoba wax, shea butter, beeswax, shellac wax, spermaceti, lanolin (wool wax), uropygial grease, ceresine, ozokerite (earth wax), paraffin waxes and microwaxes are advantageous.

Further advantageous fat and/or wax components are chemically modified waxes and synthetic waxes, such as, for example, Syncrowax® HRC (glyceryl tribehenate), and Syncrowax®AW 1 C(C₁₈₋₃₆-fatty acid) and montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleates, such as, for example, Tegosoft®CR, polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, C₂₀₋₄₀-alkyl stearate, C₂₀₋₄₀-alkyl hydroxystearoylstearate and/or glycol montanate. Furthermore, certain organosilicon compounds which have similar physical properties to the specified fat and/or wax components, such as, for example, stearoxytrimethylsilane, are also advantageous. According to the invention, the fat and/or wax components can be used either individually or as a mixture in the compositions.

Any mixtures of such oil and wax components are also to be used advantageously for the purposes of the present invention.

The oil phase is advantageously chosen from the group consisting of 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic/capric triglyceride, dicaprylyl ether.

Mixtures of octyldodecanol, caprylic/capric triglyceride, dicaprylyl ether, dicaprylyl carbonate, cocoglycerides or mixtures of C₁₂₋₁₅-alkyl benzoate and 2-ethylhexyl isostearate, mixtures of C₁₂₋₁₅-alkyl benzoate and butylene glycol dicaprylate/dicaprate and mixtures of C₁₂₋₁₅-alkyl benzoate, 2-ethylhexyl isostearate and isotridecyl isononanoate are particularly advantageous.

Of the hydrocarbons, paraffin oil, cycloparaffin, squalane, squalene, hydrogenated polyisobutene and polydecene are to be used advantageously for the purposes of the present invention.

The oil component is also advantageously chosen from the group of phospholipids. According to the invention, paraffin oil advantageous according to the invention which may be used is Merkur® white oil Pharma 40 from Merkur Vaseline, Shell Ondina® 917, Shell Ondina® 927, Shell Oil 4222, Shell Ondina®933 from Shell & DEA Oil, Pionier®6301 S, Pionier® 2071 (Hansen & Rosenthal).

Suitable cosmetically compatible oil and fat components are described in Karl-Heinz Schrader, Grundlagen und Rezepturen der Kosmetika [Fundamentals and formulations of cosmetics], 2nd Edition, Verlag Hüthig, Heidelberg, pp. 319-355, which is hereby incorporated in its entirety by reference.

The content of oils, fats and waxes is at most 30% by weight, preferably 20% by weight, further preferably at most 10% by weight, based on the total weight of the composition.

### Pigments

In one embodiment, the preparations according to the invention comprise at least one pigment. These may be colored pigments which impart color effects to the product mass or to the hair, or they may be luster effect pigments which impart luster effects to the product mass or to the hair. The color effects or luster effects on the hair are preferably temporary, i.e. they remain on the hair until the next hair wash and can be removed again by washing the hair with customary shampoos.

The pigments are present in the product mass in undissolved form and may be present in an amount of from 0.01 to 25% by weight, particularly preferably from 5 to 15% by weight. The preferred particle size is 1 to 200 μm, in particular 3 to 150 μm, particularly preferably 10 to 100 μm. The pigments are colorants which are virtually insoluble in the application medium and may be inorganic or organic. Inorganic-organic mixed pigments are also possible. Preference is given to inorganic pigments. The advantage of the inorganic pigments is their excellent stability to light, weather and temperature. The inorganic pigments may be of natural origin, prepared for example from chalk, ochre, umber, green earth, burnt sienna or graphite. The pigments may be white pigments, such as, for example, titanium dioxide or zinc oxide, black pigments, such as, for example, iron oxide black, colored pigments, such as, for example, ultramarine or iron oxide red, luster pigments, metal effect pigments, pearlescent pigments, and fluorescent or phosphorescent pigments, with preferably at least one pigment being a colored, non-white pigment.

Metal oxides, hydroxides and oxide hydrates, mixed phase pigments, sulfur-containing silicates, metal sulfides, complex metal cyanides, metal sulfates, chromates and molybdates, and the metals themselves (bronze pigments) are suitable. Titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (C177289), iron blue (ferric ferrocyanide, CI77510), carmine (cochineal) are particularly suitable.

Particular preference is given to pearlescent pigments and colored pigments based on mica which are coated with a metal oxide or a metal oxychloride such as titanium dioxide or bismuth oxychloride and, if appropriate, further color-imparting substances, such as iron oxides, iron blue, ultramarine, carmine etc. and where the color can be determined by varying the layer thickness. Such pigments are sold, for example, under the trade names Rona®, Colorona®, Dichrona® and Timiron® by Merck, Germany.

Organic pigments are, for example, the natural pigments sepia, gamboge, bone charcoal, Cassel brown, indigo, chlorophyll and other plant pigments. Synthetic organic pigments are, for example, azopigments, anthraquinoids, indigoids, dioxazine, quinacridone, phthalocyanine, isoindolinone, perylene and perinone, metal complex, alkali blue and diketopyrrolopyrrole pigments.

In one embodiment, the preparations according to the invention comprise 0.01 to 10% by weight, particularly preferably from 0.05 to 5% by weight, of at least one particulate substance. Suitable substances are, for example, substances which are solid at room temperature (25° C.) and are in the form of particles. For example, silica, silicates, aluminates, clay earths, mica, salts, in particular inorganic metal salts, metal oxides, e.g. titanium dioxide, minerals and polymer particles are suitable.

The particles are present in the composition in undissolved, preferably stably dispersed, form and, following application to the application surface and evaporation of the solvent, can be deposited in solid form.

Preferred particulate substances are silica (silica gel, silicon dioxide) and metal salts, in particular inorganic metal salts, with silica being particularly preferred. Metal salts are, for example, alkali metal or alkaline earth metal halides, such as sodium chloride or potassium chloride; alkali metal or alkaline earth metal sulfates, such as sodium sulfate or magnesium sulfate.

Suitable repellent active ingredients are compounds which are able to keep off or drive away certain animals, in particular insects, from people. These include, for example, 2-ethyl-1,3-hexanediol, N,N-diethyl-m-toluamide etc.

Suitable hyperemic substances, which stimulate the circulation of blood through the skin, are, for example, essential oils, such as dwarf pine, lavender, rosemary, juniper berry, horse chestnut extract, birch leaf extract, hay flower extract, ethyl acetate, camphor, menthol, peppermint oil, rosemary extract, eucalyptus oil, etc.

Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc.

Suitable antiphlogistics, which counteract skin irritations, are, for example, allantoin, bisabolol, dragosantol, camomile extract, panthenol, etc.

### Application Form

In a preferred embodiment, the preparations according to the invention are sprayable, for example as aerosol or pump spray preparation.

The preparations according to the invention can be used in various application forms, such as, for example, as lotion, as nonaerosol spray lotion, which is used by means of a mechanical device for spraying, as aerosol spray which is sprayed using a propellant, as aerosol foam or as nonaerosol foam, which is present in combination with a suitable mechanical device for foaming the composition, as hair cream, as hair wax, as gel, as liquid gel, as sprayable gel or as foam gel.

Use in the form of a lotion thickened with a customary thickener is also possible.

In one embodiment, the composition according to the invention is in the form of a gel, in the form of a viscous lotion or in the form of a spray gel which is sprayed using a mechanical device, and comprises at least one of the abovementioned thickeners in an amount of from preferably 0.05 to 10% by weight, particularly preferably from 0.1 to 2% by weight and has a viscosity of at least 250 mPas. The viscosity of the gel is preferably from 500 to 50 000 mPas, particularly preferably from 1000 to 15 000 mPas at 25° C.

In another embodiment, the preparation according to the invention is in the form of an O/W emulsion, a W/O emulsion or a microemulsion and comprises at least one of the abovementioned oils or waxes emulsified in water, and at least one cosmetically customary surfactant.

In a preferred embodiment, the preparation according to the invention is in the form of a spray product, either in combination with a mechanical pump spray device or in combination with at least one of the abovementioned propellants. A preferred aerosol spray additionally comprises propellants in an amount such that the total amount of the volatile organic components does not exceed 80% by weight, in particular 55% by weight of the preparation and is bottled in a pressurized container.

A nonaerosol hairspray is sprayed using a suitable mechanically operated spray device. Mechanical spray devices are understood as meaning those devices which permit the spraying of a composition without use of a propellant. A suitable mechanical spray device which may be used is, for example, a spray pump or an elastic container provided with a spray valve in which the cosmetic preparation according to the invention is bottled under pressure, where the elastic container expands and from which the composition is continuously dispensed as a result of the contraction of the elastic container from opening the spray valve.

In a further embodiment, the preparation according to the invention is in the form of a foamable product (mousse) in combination with a devices for foaming, comprises at least one customary foam-imparting substance known for this purpose, e.g. at least one foam-forming surfactant or at least one foam-forming polymer. Devices for foaming are understood as meaning those devices which permit the foaming of a liquid with or without use of a propellant. A suitable mechanical foam device which can be used is, for example, a commercially customary pump foamer or an aerosol foam head. The product is present either in combination with a mechanical pump foam device (pump foam) or in combination with at least one propellant (aerosol foam) in an amount of from preferably 1 to 20% by weight, in particular from 2 to 10% by weight. Propellants are, for example, chosen from propane, butane, dimethyl ether and fluorinated hydrocarbons.

The invention thus provides a cosmetic, preferably hair cosmetic preparation in the form of a spray product, where the preparation is present either in combination with a mechanical pump spray device or in combination with at least one propellant chosen from the group consisting of propane, butane, dimethyl ether, fluorinated hydrocarbons and mixtures thereof.

The composition is foamed directly prior to use and incorporated into the hair as foam and can then be rinsed out or left in the hair without rinsing out.

A formulation preferred according to the invention for aerosol hair foams comprises
i) 0.1 to 10% by weight of at least one polymer A,
ii) 55 to 99.8% by weight of water and alcohol,
iii) 5 to 20% by weight of a propellant,
iv) 0.1 to 5% by weight of an emulsifier,
v) 0 to 10% by weight of further constituents, where the total amount of VOC is at most 80% by weight and preferably 55% by weight.

Emulsifiers which can be used are all emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers can, for example, be chosen from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
i) 0.1 to 10% by weight of polymer A,

ii) 80 to 99.85% by weight of water and alcohol,
iii) 0 to 3% by weight, preferably 0.05 to 2% by weight, of a gel former,
iv) 0 to 20% by weight of further constituents where the total amount of VOC is at most 80% by weight and preferably 55% by weight.

When preparing gels based on the polymers A, customary gel formers can be used, for example, in order to establish specific rheological or other applications-related properties. Gel formers which can be used are all gel formers customary in cosmetics. These include slightly crosslinked polyacrylic acid, for example carbomer (INCI), cellulose derivatives, e.g. hydroxypropylcellulose, hydroxyethylcellulose, canionically modified celluloses, polysaccharides, e.g. xanthan gum, caprylic/capric triglyceride, sodium acrylate copolymers, polyquaternium-32 (and) Paraffinum Liquidum (INCI), sodium acrylate copolymers (and) Paraffinum Liquidum (and) PPG-1 trideceth-6, acrylamidopropyltrimonium chloride/acrylamide copolymers, steareth-10 alkyl ether acrylate copolymers, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44. Crosslinked homopolymers of acrylic acid suitable as gel formers are commercially available, for example, under the name Carbopol® (Noveon). Preference is also given to hydrophobically modified crosslinked polyacrylate polymers, such as Carbopol®Ultrez 21 (Noveon). Further examples of anionic polymers suitable as gel formers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are commercially available as Aculyn® (Rohm and Haas).

In a further embodiment, the preparation according to the invention is in the form of a hair wax, i.e. it has wax-like consistency and comprises at least one of the abovementioned waxes in an amount of from preferably 0.5 to 30% by weight, and if appropriate further water-insoluble substances. The wax-like consistency is preferably characterized in that the needle penetration number (unit of measurement 0.1 mm, test weight 100 g, test time 5 s, test temperature 25° C.; in accordance with DIN 51 579) is greater than or equal to 10, particularly preferably greater than or equal to 20 and that the solidification point of the product is preferably greater than or equal to 30° C. and less than or equal to 70° C., is particularly preferably in the range from 40 to 55° C. Suitable waxes and water-insoluble substances are, in particular, emulsifiers with a HLB value below 7, silicone oils, silicone waxes, waxes (e.g. wax alcohols, wax acids, wax esters, and in particular natural waxes, such as beeswax, carnauba wax etc.), fatty alcohols, fatty acids, fatty acid esters or hydrophilic waxes, such as, for example, high molecular weight polyethylene glycols with a molecular weight of from 800 to 20 000 g/mol, preferably from 2000 to 10 000 g/mol.

If the cosmetic, preferably hair cosmetic, preparation according to the invention is in the form of a hair lotion, then it is present as an essentially non-viscous or low-viscosity, flowable solution, dispersion or emulsion with a content of at least 10% by weight, preferably 20 to 95% by weight, of a cosmetically compatible alcohol. Alcohols which can be used are, in particular, the lower alcohols having 1 to 4 carbon atoms customarily used for cosmetic purposes, e.g. ethanol and isopropanol.

If the hair cosmetic preparation according to the invention is in the form of a hair cream, then it is preferably in the form of an emulsion and comprises either additionally viscosity-imparting ingredients in an amount of from 0.1 to 10% by weight, or the required viscosity and creamy consistency is built up through micelle formation with the help of suitable emulsifiers, fatty acids, fatty alcohols, waxes etc. in the customary way.

The polymers A according to the invention can be used as conditioners in cosmetic preparations.

The polymers A according to the invention can preferably be used in shampoo formulations as setting and/or conditioning agents. Preferred shampoo formulations comprise
i) 0.05 to 10% by weight of at least one polymer A,
ii) 25 to 94.95% by weight of water,
iii) 5 to 50% by weight of surfactants,
iv) 0 to 5% by weight of a further conditioning agent,
v) 0 to 10% by weight of further cosmetic constituents.

In the shampoo formulations, all of the anionic, neutral, amphoteric or cationic surfactants customarily used in shampoos can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate are suitable.

Suitable amophoteric surfactants are, for example, alkylbetaines, alkylaminopropylbetaines, alkylsulfobetaines, alkyl glycinates, alkyl carboxyglycinates, alkyl amphoacetates or propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. In addition, alkylamine oxides, mono- or diallkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioners can be used in combination with the polymers A. These include, for example, the abovementioned cationic polymers with the INCI name Polyquaternium, in particular copolymers of vinylpyrrolidone/N-vinylimidazolium salts (Luviquat®FC, Luviquat®HM, Luviquat®MS, Luviquat®Care, Luviquat®Ultracare), copolymers of N-vinylpyrrolidone/dimethylaminoethyl methacrylate, quaternized with diethyl sulfate (Luviquat®PQ 11), copolymers of N-vinylcaprolactam/N-vinylpyrrolidone/N-vinyl-imidazolium salts (Luviquat®Hold); cationic cellulose derivatives (polyquaternium-4 and -10), acrylamide copolymers (polyquaternium-7). It is also possible to use protein hydrolysates, and conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and aminofunctional silicone compounds, such as amodimethicones (CTFA). In addition, cationic guar derivatives, such as guar hydroxypropyltrimonium chloride (INCI) can be used.

The examples below serve to illustrate the subject-matter of the invention in more detail without limiting the invention thereto.

### Measurement Methods

### Determination of the K Value

The K values are measured in accordance with Fikentscher, Cellulosechemie, Vol. 13, p. 58 to 64 (1932) at 25° C. in ethanol or N-methylpyrrolidone (NMP) solution and are a measure of the molecular weight. The ethanol or NMP solutions of the polymers each comprise 1 g of polymer A in 100 ml of solution.

If the polymers are in the form of aqueous dispersions, corresponding amounts of the dispersion are topped up with ethanol to 100 ml depending on the polymer content of the dispersion, so that the concentration is 1 g in 100 ml.

The K value is measured in a micro-Ubbelohde capillary type M Ic from Schott.

### Determination of the Droplet Size Distribution (DSD) by Means of Malvern® Scattered Light Analysis

The droplet size distribution was determined using particle size measurement system for detecting liquid aerosols “Malvern®Master Sizer X” (Malvern Instruments Inc., Southborough Mass., USA).

### Measurement Principle:

The measurement system is based on the method of laser light diffraction at the particle, which is suitable not only for spray analysis (aerosols, pump sprays), but also for determining the size of solids, suspensions and emulsions in the size range from 0.1 μm to 2000 μm.

A particle collective (=droplet) is illuminated by a laser. At each droplet, some of the incident laser light is scattered. This light is captured on a multielement detector and the corresponding light energy distribution is determined. This data is used to calculate the corresponding particle distribution using the evaluation software.

### Procedure:

The aerosols were sprayed at a distance of 29.5 cm from the laser beam. The spray cone was at right angles to the laser beam.

Before each measurement, the aerosol cans were attached to a firmly installed holding device, thus meaning that all of the aerosols to be tested were measured at exactly the same distance.

Before the actual particle measurement, a “background measurement” was carried out. By doing so, the effects of dust and other contaminants within the measurement range were eliminated.

The aerosol was then sprayed into the test space. The entire particle volume was detected for a test period of 2s and evaluated.

### Evaluation:

The evaluation comprises a tabular depiction over 32 class widths from 0.5 μm to 2000 μm and additionally a graphical depiction of the particle size distribution.

Since the spray experiments are an approximately uniform distribution, the mean diameter D (v, 0.5) is given. This numerical value indicates that 50% of the total particle volume measured is below this value.

For readily sprayable aerosol systems in the cosmetics sector, this value is in the range from 30 μm to 80 μm, depending on the polymer content, geometry of the valve and actuator, solvent ratio and amounts of propellant gas.

Determination of the Setting (Flexural Rigidity):

The setting of polymeric film formers was measured not only by a subjective assessment (hand test), but also physically by measuring the flexural rigidity of thin hair swatches (each about 3 g and 24 cm in length). For this, the weighed, dry hair swatches were dipped into the 3.0% strength by weight polymer solution (solvent: ethanol/water 55:45 w/w), uniform wetting of the hair swatches and distribution of the polymer solution being ensured by dipping and removing the swatches three times and then squeezing them between filter paper. The excess film former solution was then squeezed out between thumb and forefinger and the hair swatches were the swatches shaped by hand so that they had a round cross section. They were dried overnight in a climatically controlled room at 20° C. and 65% relative humidity. The tests were carried out in the climatically controlled room at 20° C. and 65% relative humidity using a stress/strain testing device. The hair swatch was placed symmetrically at the ends on two cylindrical rolls of the sample holder. In exactly the middle, the swatch was then bent from above using a rounded punch ca. 40 mm (breakage of the polymer film). The force required for this (Fmax) was determined using a weighing cell (50 N). Here, one measurement value represents the arithmetic mean from the individual measurements on 5 to 10 identically treated hair swatches. The values ascertained in this way were placed in relation to those for a standard commercial comparison polymer (Amphomer® LV-71) and given in %.

### Determination of the Ability to be Washed Out:

A hair swatch treated with polymer analogously to the determination of the setting was washed in a ca. 37° C.-hot Texapon®NSO solution (6 ml of Texapon®NSO (28% strength) in 1 l of warm water) for ca. 15 seconds by dipping it in and squeezing it five times. The hair swatch was then rinsed until clear and treated again in the same way.

The hair swatch was then squeezed thoroughly on filter paper and left to dry overnight. The dry hair swatch was put in rollers and analyzed for residues.

### Determination of the Curl Retention

### Basic Formulation: (Aerosol Hairspray)

| | |
|---|---|
| 5% by weight | of active ingredient of polymer to be tested (100% neutr. |
| | with AMP) |
| 15% by weight | of ethanol |
| 40% by weight | of water |
| 40% by weight | of dimethyl ether. |

To determine the curl retention, hair swatches ca. 2 g in weight and 15.5 cm in length and comprising mid-brown, Caucasian human hair were used.

### Treatment of the Hair Swatches:

The hair swatches were washed twice with an aqueous Texapon®NSO solution. The hair swatches were then rinsed with warm water until no more foaming was evident and after-rinsed with demineralized water, combed and laid to dry on filter paper.

To prepare a waterwave, the hair swatches are placed for 15 minutes to swell in a solution of ethanol and water (1:1).

The hair swatch was carefully combed before the curl preparation. The hair swatch was attached to a plexiglass rod using a rubber band. It was then combed and wound in the shape of a spiral. Using a cotton cloth and rubber band, the curl was firmly fixed and dried overnight at 70° C. The cooled curl retention swatches were carefully opened and slipped off the plexiglass rod without deforming the waterwave. From a distance of 15 cm, 1.8 g of the aerosol hairspray prepared as mentioned above were sprayed uniformly onto the curl. The curl was rotated evenly during this. In the horizontal position, the curls were dried for 1 h at room temperature. After drying, the curls were secured in a support. Using a ruler, the starting length of the curls at the start was read off and the length extension during humid storage was monitored. After storage for 5 h at 25° C. and 90% relative humidity in the climatically controlled chamber, the length which the curl had reached was read off again and the curl retention was calculated according to the following equation:

$$Curl Retention in \% = \frac{L - {L}_{t}}{L - {L}_{o}} * 100$$

L Length of the hair (15.5 cm)
L₀ Length of the hair curl after drying
Lₜ=Length of the hair curl after climatic treatment

The mean value from the 5 individual measurements was given as curl retention.

### Determination of the Stickiness

Firstly, a clear, 20% strength by weight ethanolic or ethanolic/aqueous solution of the polymer to be characterized was prepared. In order to obtain a clear solution it was sometimes necessary to neutralize the polymer. A doctor knife (120 μm slit width) was then used to apply a film of the polymer from the ethanolic or ethanolic/aqueous solution on a glass plate. This rectangular glass plate had a length of ca. 20 cm and a width of ca. 6.5 m. The polymer film applied thereto had in each case a length of ca. 16 to 18 cm and a width of ca. 5.5 cm.

The film was then dried in the air for ca. 10 hours and then stored in a climatically controlled cabinet for a further 12 hours at 20° C. and 80% relative humidity.

Then, under these conditions, in the climatically controlled cabinet, a plastic carbon ribbon located on a round rubber punch (diameter 400 mm, Shore A hardness 60±5) was pressed onto the polymer film with a force of about 250 N for 10 seconds.

The amount of black pigment which remains adhering to the polymer film after the punch has been removed corresponds to the stickiness of the film. A visual assessment of the black coloration of the film was made. The assessment scale ranges from 0 to 5, where 0 is not sticky and 5 is very considerably sticky.

### Determination of the Appearance of the Aerosol Formulation

Preparations comprising 5% by weight of the particular polymer neutralized with AMP, 40% by weight of DME, 15% by weight of ethanol and 40% by weight of water were poured into a transparent glass aerosol container. The clarity of the resulting liquid/propellant gas mixture was then assessed visually.

### EXAMPLES

The following examples illustrate the invention without limiting it.

Unless indicated otherwise, the percentages are percentages by weight.

### Abbreviations Used:

t-BA Tert-butyl acrylate
MAA Methacrylic acid
AA Acrylic acid
ITS Itaconic acid
EA Ethyl acrylate
MMA Methyl methacrylate
EMA Ethyl methacrylate
t-BMA Tert-butyl methacrylate
i-BMA Isobutyl methacrylate
CD completely demineralized

### Preparation of the Polymers

To prepare the polymers A according to the invention, the following polyester acrylates were used as component c):

### Polyether Acrylate 1:

Preparation according to example 1 of EP-A 0 279 303, p. 3

Dispersion analogous to DE 2853921, p. 17, example 2.
Polyester acrylate 1 was used as 50% strength by weight dispersion, referred to below for short as polyester acrylate 1.

### Polyester Acrylate 2:

Preparation according to comparison example 1 of EP-A 0 279 303, p. 5

Dispersion analogous to DE 2853921, p. 17, example 2.
Polyester acrylate 2 was used as 50% strength by weight dispersion, referred to below for short as polyester acrylate 2

### Polyether Acrylate 3:

Preparation according to EP-A 0 279 303, p. 3, example 1.

### Example 1

### Preparation of Polymer 1 (Solution Polymerization in Ethanol)

The following feeds were prepared at 20° C. with stirring:

### Feed 1:

| | |
|---|---|
| 196 g | of EMA |
| 72 g | of MAA |
| 10 g | of polyether acrylate 1 |
| 200 g | of ethanol |

### Feed 2:

| | |
|---|---|
| 7 g | of Wako ® V 59 |
| 50 g | of ethanol |

At 20° C., a mixture of 300 g of ethanol, 15% of the total amount of feed 1, and 15% of the total amount of feed 2 were prepared. The mixture was heated to 78° C. under atmospheric pressure. After 78° C. had been reached, feed 1 and feed 2 were started at the same time. Feed 1 was metered in over the course of 3 h, and feed 2 was metered in over the course of 4 h with a constant feed stream. The reaction mixture was maintained at 78° C. throughout the entire feed. When feed 2 was complete, the reaction mixture was kept at 78° C. for a further 2 h, then cooled to room temperature.

### Comparative Example C1

In a 2 l polymerization vessel fitted with stirrer and heating and cooling devices,

| | |
|---|---|
| 400 g | of deionized water |
| 0.6 g | of a 15% strength by weight aqueous solution of sodium |
| | lauryl sulfate |
| 35 g | of feed II (see below) |

were initially introduced at a temperature of from 20 to 25° C. and heated to 45° C. with stirring and under a nitrogen atmosphere. After the temperature had been reached, feed 1 was added over the course of 5 minutes. The mixture was then heated to 80° C. and, while stirring and maintaining the reaction temperature, feed II was metered in with constant feed streams over the course of 2.5 hours. When the feeds were complete, the reaction mixture was stirred for a further hour at 80° C. and then cooled to 60° C. While maintaining the temperature of 60° C., feed III was added. The mixture was then cooled to 35° C. and, while maintaining the temperature, feed IV was added.

### Feed I:

| | |
|---|---|
| 6 g | of 7% strength by weight aqueous solution of sodium persulfate in |
| | deionized water |

Feed II: aqueous monomer emulsion prepared from

| | |
|---|---|
| 204 g | of deionized water |
| 8 g | of a 15% strength by weight aqueous solution of sodium lauryl |
| | sulfate, |
| 40 g | of a 25% strength by weight solution of Tween ™ 80 in CD water, |
| 297 g | of methyl methacrylate, |
| 99 g | of methacrylic acid, |
| 2.4 g | of n-dodecyl mercaptan. |

The total amount of the 15% strength by weight aqueous solution of sodium lauryl sulfate was added to the initial charge of deionized water with stirring. The corresponding amounts of

Methyl methacrylate,
25% strength by weight solution of Tween™ 80 in CD water,
Methacrylic acid and
n-dodecyl mercaptan
were added in the order given to the homogeneous solution, which was further stirred.

### Feed III:

| | | |
|---|---|---|
| 4 g | of 30% strength by weight solution of hydrogen peroxide in | 0.3 |
| | deionized water | |

### Feed IV:

| | |
|---|---|
| 40 g | of 10% strength by weight solution of ammonium |
| | hydrogencarbonate in deionized water |

Examples 2, 3, 4, 10, 11, 12, 13 and comparison example 3 (C3) in the table below were prepared analogously to example 1. Polymer C1 was prepared by emulsion polymerization analogously to the described example.

| Polymer | Monomers | Weight ratio |
|---|---|---|
| C1 | MMA/MAA | 75/25 |
| C3 | MMA/MAA | 75/25 |
| 1 | EMA/MAA/polyether acrylate 1 | 70.5/26/3.5 |
| 2 | MMA/MAA/polyether acrylate 1 | 71/25/4 |
| 3 | t-BA/MAA/polyether acrylate 1 | 71.5/25/3.5 |
| 4 | t-BA/MAA/polyester acrylate 2 | 71.5/25/3.5 |
| 10 | MMA/ITS/polyether acrylate 1 | 71/25/4 |
| 11 | MMA/AA/ITS/polyether acrylate 1 | 71/10/15/5 |
| 12 | MMA/MAA/polyether acrylate 1 | 67/26/7 |
| 13 | EMA/MAA/AA/polyether acrylate 1 | 67/14/12/7 |

Polymers with the following compositions can, for example, also be prepared analogously to the above examples:

| | |
|---|---|
| t-BA/MAA/polyether acrylate 3 | 71.5/25/3.5 |
| i-BMA/MAA/polyether acrylate 1 | 71.5/25/3.5 |
| t-BA/HEMA/MAA/polyether acrylate 1 | 42.5/28/25/3.5 |
| MMA/AA/MAA/polyether acrylate 1 | 71/13/12/4 |

### Application Properties of the Polymers a According to the Invention

| | | | Setting [% | |
|---|---|---|---|---|
| | | | compared to | DSD |
| | Clarity as | Ability to be | Amphomer ® | Malvern |
| Example | aerosol | washed out | LV71]^{a)} | [μm] |
| C1 | almost clear | poor | 90 | 70 |
| C3 | slightly cloudy | — | — | — |
| 1 | clear | still good | 75 | 50 |
| 2 | almost clear | good | 115 | 50 |
| 3 | clear | good | 85 | 70 |
| 4 | clear | good | 90 | 70 |
| 10 | clear | good | 75 | 33 |
| 11 | almost clear | good | 91 | 65 |
| 12 | clear | good | 80 | 33 |
| 13 | clear | good | 65 | 40 |

^{a)}VOC 55 Aerosol: 5% of the respective polymer, neutralized completely with AMP, 40% of DME, 15% of ethanol, 40% of water;
Spray device:
Actuator: Kosmos .020D Wirbel .018″ 21-6443-20 (Precision Valve),
Valve: DPV 33876 (Precision Valve)

### II) Application Examples

Unless stated otherwise, all of the polymers containing acid groups used are 100% neutralized with 2-amino-2-methylpropanol (AMP). “Water ad 100” means that the amount of water necessary to reach a total amount of 100% by weight is added to the particular preparation.

The quantitative data % are % by weight unless determined in some other way.

The abbreviation “q.s.” means “quantum satis”, i.e. add as much of an ingredient as is necessary to achieve a desired effect.

The designation (solid) means that the amount of polymer used is calculated on the basis of the solid content if the polymer is in solution form.

### Example 1a

VOC 55 aerosol hairspray

| | [%] |
|---|---|
| Polymer from example no. 2 (solid) | 5.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4, 10 to 13 according to the invention. In each case, a VOC 55 aerosol hairspray with good properties is obtained.

### Example 1b

VOC 55 aerosol hairspray

| | [%] |
|---|---|
| Polymer from example No. 2 (solid) | 3.00 |
| Balance ® 0/55 (National Starch) | 2.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4, 10 to 13 according to the invention. In each case, a VOC 55 aerosol hairspray with good properties is obtained.

### Example 1c

VOC 55 aerosol hairspray

| | [%] |
|---|---|
| Polymer from example No. 2 (solid) | 3.00 |
| Acudyne ® 180 (Rohm&Haas) | 1.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 1d

VOC 55 aerosol hairspray

| | [%] |
|---|---|
| Polymer from example No. 2 (solid) | 3.00 |
| Amphomer ® LV 71 (National Starch) | 2.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 1e

VOC 55 aerosol hairspray

| | [%] |
|---|---|
| Polymer from example No. 2 (solid) | 3.00 |
| Acudyne ® DHR (Rohm&Haas) | 1.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 1e

VOC 55 aerosol hairspray

| | [%] |
|---|---|
| Polymer from example No. 2 (solid) | 3.00 |
| Eastman ® AQ 48 (Eastman-Kodak) | 2.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 1f

VOC 55 aerosol hairspray

| | [%] |
|---|---|
| Polymer from example No. 2 (solid) | 3.00 |
| Resyn ® 28-2930 (National Starch) | 2.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 1g

VOC 55 aerosol hairspray

| | [%] |
|---|---|
| Polymer from example No. 2 (solid) | 3.00 |
| Balance ® 47 (National Starch) | 2.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 1h

VOC 55 aerosol hairspray

| | [%] |
|---|---|
| Polymer from example No. 2 (solid) | 3.00 |
| Aquaflex ® SF-40 (ISP) | 1.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 1i

VOC 55 aerosol hairspray

| | [%] |
|---|---|
| Polymer from example No. 2 (solid) | 3.00 |
| DynamX ® (ISP) | 1.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2a

VOC 55 aerosol hairspray

| | [%] |
|---|---|
| Polymer from example No. 2 (solid) | 5.00 |
| Dimethyl ether | 35.00 |
| Propane/butane | 5.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2b

VOC 55 aerosol hairspray

| | [%] |
|---|---|
| Polymer from example No. 2 (solid) | 3.00 |
| Balance ® 0/55 (National Starch) | 2.00 |
| Dimethyl ether | 35.00 |
| Propane/butane | 5.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2c

VOC 55 aerosol hairspray

| | [%] |
|---|---|
| Polymer from example No. 2 (solid) | 3.00 |
| Acudyne ® 180 (Rohm&Haas) | 1.00 |
| Dimethyl ether | 40.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2d

VOC 55 aerosol hairspray

| | [%] |
|---|---|
| Polymer from example No. 2 (solid) | 3.00 |
| Amphomer ® LV 71 (National Starch) | 2.00 |
| Dimethyl ether | 35.00 |
| Propane/butane | 5.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2e

VOC 55 aerosol hairspray

| | [%] |
|---|---|
| Polymer from example No. 2 (solid) | 3.00 |
| Acudyne ® DHR (Rohm&Haas) | 1.00 |
| Dimethyl ether | 35.00 |
| Propane/butane | 5.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2e

VOC 55 aerosol hairspray

| | [%] |
|---|---|
| Polymer from example No. 2 (solid) | 3.00 |
| Eastman ® AQ 48 (Eastman-Kodak) | 2.00 |
| Dimethyl ether | 35.00 |
| Propane/butane | 5.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2f

VOC 55 aerosol hairspray

| | [%] |
|---|---|
| Polymer from example No. 2 (solid) | 3.00 |
| Resyn ® 28-2930 (National Starch) | 2.00 |
| Dimethyl ether | 35.00 |
| Propane/butane | 5.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2g

VOC 55 aerosol hairspray

| | [%] |
|---|---|
| Polymer from example No. 2 (solid) | 3.00 |
| Balance ® 47 (National Starch) | 2.00 |
| Dimethyl ether | 35.00 |
| Propane/butane | 5.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2h

VOC 55 aerosol hairspray

| | [%] |
|---|---|
| Polymer from example No. 2 (solid) | 3.00 |
| Aquaflex ® SF-40 (ISP) | 1.00 |
| Dimethyl ether | 35.00 |
| Propane/butane | 5.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 2i

VOC 55 aerosol hairspray

| | [%] |
|---|---|
| Polymer from example No. 2 (solid) | 3.00 |
| DynamX ® (ISP) | 1.00 |
| Dimethyl ether | 35.00 |
| Propane/butane | 5.00 |
| Ethanol | 15.00 |
| Water | ad 100 |

Further additives: silicone, perfume, antifoam, UV absorber

The example can be repeated in each case with the polymers 1 and 3, 4 and 10 to 13 according to the invention. In each case, a VOC 55 aerosol hairspray with good properties is obtained.

### Example 3a

Aerosol hairspray with fluorocarbon propellants

| | [%] |
|---|---|
| Polymer from example No. 2 (solid) | 5.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 3b

VOC 55 aerosol hairspray

| | [%] |
|---|---|
| Polymer from example No. 2 (solid) | 3.00 |
| Balance ® 0/55 (National Starch) | 2.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 3c

VOC 55 aerosol hairspray

| | [%] |
|---|---|
| Polymer from example No. 2 (solid) | 3.00 |
| Acudyne ® 180 (Rohm&Haas) | 1.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 3d

VOC 55 aerosol hairspray

| | [%] |
|---|---|
| Polymer from example No. 2 (solid) | 3.00 |
| Amphomer ® LV 71 (National Starch) | 2.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 3e

VOC 55 aerosol hairspray

| | [%] |
|---|---|
| Polymer from example No. 2 (solid) | 3.00 |
| Acudyne ® DHR (Rohm&Haas) | 1.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 3e

VOC 55 aerosol hairspray

| | [%] |
|---|---|
| Polymer from example No. 2 (solid) | 3.00 |
| Eastman ® AQ 48 (Eastman-Kodak) | 2.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 3f

VOC 55 aerosol hairspray

| | [%] |
|---|---|
| Polymer from example No. 2 (solid) | 3.00 |
| Resyn ® 28-2930 (National Starch) | 2.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 3g

VOC 55 aerosol hairspray

| | [%] |
|---|---|
| Polymer from example No. 2 (solid) | 3.00 |
| Balance ® 47 (National Starch) | 2.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 3h

VOC 55 aerosol hairspray

| | [%] |
|---|---|
| Polymer from example No. 2 (solid) | 3.00 |
| Aquaflex ® SF-40 (ISP) | 1.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 31

VOC 55 aerosol hairspray

| | [%] |
|---|---|
| Polymer from example No. 2 (solid) | 3.00 |
| DynamX ® (ISP) | 1.00 |
| Ethanol abs. | ad 100 |
| HFC 152A | 40.00 |

Further additives: silicone, perfume, antifoam, UV absorber

The example can be repeated in each case with the polymers 1 and 3, 4 and 10 to 13 according to the invention. In each case, an aerosol hairspray with good properties is obtained.

### Example 4

Aerosol hairspray with fluorocarbon propellants

| | [%] |
|---|---|
| Polymer from example No. 2 (solid) | 5.00 |
| Dist. water | ad 100 |
| HFC 152A | 10.00 |
| Dimethyl ether | 30.00 |
| Ethanol abs. | 30.00 |

Further additives: silicone, perfume, antifoam, UV absorber

The example can be repeated in each case with the polymers 1-11 and 13-15. In each case, an aerosol hairspray with good properties is obtained.

### Example 5

VOC 55 aerosol hairspray

| | [%] |
|---|---|
| Polymer from example No. 2 (solid) | 3.00 |
| Ultrahold ® Strong (solid, BASF) | 1.00 |
| Dimethyl ether | 40.00 |
| Ethanol | 15.00 |
| +AMP | to pH 8.3 |
| Water | ad 100 |

Further additive: silicone, perfume, antifoam

The example can be repeated in each case with the polymers 1 and 3, 4 and 10 to 13 according to the invention. In each case, a VOC 55 aerosol hairspray with good properties is obtained.

### Example 6

VOC 55 aerosol hairspray

| | [%] |
|---|---|
| Polymer from example No. 2 (solid) | 3.00 |
| Luvimer ® Pro55 (solid, BASF) | 1.00 |
| Dimethyl ether | 40.00 |
| Ethanol | 15.00 |
| Water | ad 100 |

Further additive: silicone, perfume, antifoam

The example can be repeated in each case with the polymers 1 and 3, 4 and 10 to 13 according to the invention. In each case, a VOC 55 aerosol hairspray with good properties is obtained.

### Example 7

VOC 55 aerosol hairspray

| | [%] |
|---|---|
| Polymer from example No. 2 (solid) | 3.00 |
| Luvimer ® P.U.R (solid, BASF) | 1.00 |
| Dimethyl ether | 40.00 |
| Ethanol | 15.00 |
| Water | ad 100 |

Further additive: silicone, perfume, antifoam

The example can be repeated in each case with the polymers 1 and 3, 4 and 10 to 13 according to the invention. In each case, a VOC 55 aerosol hairspray with good properties is obtained.

### Example 8

VOC 55 aerosol hairspray

| | [%] |
|---|---|
| Polymer from example No. 2 (solid) | 3.00 |
| Resyn ® 28-2930 (solid, National Starch) | 1.00 |
| Dimethyl ether | 40.00 |
| Ethanol | 15.00 |
| Water | ad 100 |

Further additive: silicone, perfume, antifoam

The example can be repeated in each case with the polymers 1 and 3, 4 and 10 to 13 according to the invention. A VOC 55 aerosol hairspray with good properties is likewise obtained.

### Example 9

VOC 55 aerosol hairspray

| | [%] |
|---|---|
| Polymer from example No. 2 (solid) | 2.00 |
| Stepanhold ® R-1*⁾ (Stepan Chemical Co.) | 1.00 |
| Dimethyl ether | 40.00 |
| Ethanol | 15.00 |
| +AMP | to pH 8.3 |
| Water | ad 100 |

Further additive: silicone, perfume, antifoam
*⁾Stepanhold ® R-1 = poly(vinylpyrrolidone/ethyl methacrylate/methacrylic acid)

The example can be repeated in each case with the polymers 1 and 3, 4 and 10 to 13 according to the invention. In each case, a VOC 55 aerosol hairspray with good properties is obtained.

### Example 10

VOC 55 hand pump spray

| | [%] |
|---|---|
| Polymer from example No. 2 (solid) | 7.00 |
| Ethanol | 55.00 |
| Water | ad 100 |

Further additive: silicone, perfume, antifoam

The example can be repeated in each case with the polymers 1 and 3, 4 and 10 to 13 according to the invention. In each case, a VOC 55 hand pump spray with good properties is obtained.

### Example 11

VOC 80 aerosol hairspray

| | [%] |
|---|---|
| Polymer from example No. 2 (solid) | 12.00 |
| Dimethyl ether | 40.00 |
| Ethanol | 40.00 |
| Water | ad 100 |

Further additive: silicone, perfume, antifoam

The example can be repeated in each case with the polymers 1 and 3, 4 and 10 to 13 according to the invention. In each case, a VOC 80 aerosol hairspray with good properties is obtained.

### Example 11

| Aqueous hand pump spray | [%] |
|---|---|
| Polymer from example No. 2 (solid) | 4.00 |
| Luviset ® Clear*⁾ (solid) | 1.00 |
| Water | ad 100 |

Further additive: Water-soluble silicone, perfume, antifoam.
*⁾Luviset ® Clear: poly(vinylpyrrolidone/methacrylamide/vinylimidazole), BASF

The example can be repeated in each case with the polymers 1 and 3, 4 and 10 to 13 according to the invention. In each case, an aqueous hand pump spray with good properties is obtained.

### Example 12

| Aqueous/ethanolic setting solution | [%] |
|---|---|
| Polymer from example No. 2 (solid) | 7.00 |
| Dist. water | ad 100 |
| Ethanol | 52.00 |

Further additive: silicone, perfume, antifoam

The example can be repeated in each case with the polymers 1 and 3, 4 and 10 to 13 according to the invention. In each case, a setting lotion with good properties is obtained.

### Example 13

| Ethanolic setting solution | [%] |
|---|---|
| Polymer from example No. 2 (solid) | 7.0 |
| Ethanol | ad 100 |

Further additive: silicone, perfume, antifoam . . .

The example can be repeated in each case with the polymers 1 and 3, 4 and 10 to 13 according to the invention. In each case, a setting lotion with good properties is obtained.

### Example 14

| Hair gel with Aculyn 28: | [%] |
|---|---|
| Phase 1: | |
| Polymer from example No. 2 (solid) | 6.00 |
| Aminomethylpropanol (38% strength solution) | 1.0 |
| Water, dist. | ad 50 |
| Phase 2: | |
| Aculyn 28 (1% strength aqueous suspension) | 50.00 |

Further additive: Preservative, soluble ethoxylated silicone, perfume . . .

### Preparation:

Phases 1 and 2 are weighed in separately and homogenized. Phase 2 is then slowly stirred into phase 1. An essentially clear, stable gel is formed.

The example can be repeated in each case with the polymers 1 and 3, 4 and 10 to 13 according to the invention. In each case, a hair gel with Aculyn 28 with good properties is obtained.

### Example 15

| Hair gel with hydroxyethylcellulose: | [%] |
|---|---|
| Phase 1: | |
| Polymer from example No. 2 (solid) | 6.00 |
| Water, dist. | ad 50 |
| Phase 2: | |
| Natrosol HR 250 (5% strength solution) | 50.00 |
| Hydroxyethylcellulose (Hercules) | |

Further additive: Preservative, soluble ethoxylated silicone, perfume

### Preparation:

Phases 1 and 2 are weighed in separately and homogenized. Phase 2 is then slowly stirred into phase 1. An essentially clear, stable gel is formed.

The example can be repeated in each case with the polymers 1 and 3, 4 and 10 to 13 according to the invention. In each case, a hair gel with hydroxyethylcellulose with good properties is obtained.

### Example 16

| Foam conditioner | [%] |
|---|---|
| Polymer from example No. 2 (solid) | 0.50 |
| Cremophor ® A 25 (Ceteareth 25/BASF) | 0.20 |
| Comperlan ® KD (coamide DEA/Henkel) | 0.10 |
| Propane/butane | 10.00 |
| Water | ad 100 |

Further additive: perfume, preservative

Preparation: Weigh in and Dissolve with Stirring. Bottle and Add Propellant Gas.

The example can be repeated in each case with the polymers 1 and 3, 4 and 10 to 13 according to the invention. In each case, a foam conditioner with good properties is obtained.

### Example 17

| Conditioner shampoo: | | [%] |
|---|---|---|
| A) | Texapon ® NSO 28% strength (sodium laureth | 50.00 |
| | sulfate/Henkel) | |
| | Comperlan ® KS (coamide DEA/Henkel) | 1.00 |
| | Polymer from example No. 2 (solid) | 3.00 |
| | q.s. Perfume oil | |
| B) | Water | 44.5 |
| | Sodium chloride | 1.5 |
| | q.s. Preservative | |

### Preparation:

Phases 1 and 2 are weighed in separately and homogenized. Phase 2 is then slowly stirred into phase 1. An essentially clear, stable gel is formed.

The example can be repeated in each case with the polymers 1 and 3, 4 and 10 to 13 according to the invention. In each case, a conditioner shampoo with good properties is obtained.

### Example 18

Standard O/W cream:

| | [%] | CTFA name |
|---|---|---|
| Oil phase: | | |
| Cremophor ® A6 | 3.5 | Ceteareth-6 (and) Stearyl |
| | | Alcohol |
| Cremophor ® A25 | 3.5 | Ceteareth-25 |
| Glycerol monostearate s.e. | 2.5 | Glyceryl stearate |
| Paraffin oil | 7.5 | Paraffin Oil |
| Cetyl alcohol | 2.5 | Cetyl Alcohol |
| Luvitol ® EHO | 3.2 | Cetearyl Octanoate |
| Vitamin E acetate | 1.0 | Tocopheryl Acetate |
| Nip-Nip | 0.1 | Methyl- and Propyl-4- |
| | | hydroxybenzoate (7:3) |
| Water phase: | | |
| Polymer from example No. 2 (solid) | 0.6 | |
| Water | 77.0 | |
| 1,2-Propylene glycol | 1.5 | propylene glycol |
| Germall II | 0.1 | Imidazolidinylurea |

### Preparation:

The oil and water phases are weighed in separately and homogenized at a temperature of about 80° C. The water phase is then slowly stirred into the oil phase and slowly cooled to room temperature with stirring.

The example can be repeated in each case with the polymers 1 and 3, 4 and 10 to 13 according to the invention. In each case, a standard O/W cream with good properties is obtained.

### Example 19

### Liquid Makeup

A

| | |
|---|---|
| 1.70 | Glyceryl stearate |
| 1.70 | Cetyl alcohol |
| 1.70 | Ceteareth-6 |
| 1.70 | Ceteareth-25 |
| 5.20 | Caprylic/capric triglyceride |
| 5.20 | Mineral oil |

B

| | |
|---|---|
| q.s. | Preservative |
| 4.30 | Propylene glycol |
| 2.50 | Polymer from example 2 (solid) |
| ad 100 | Dist. water |

C

| | |
|---|---|
| q.s. | Perfume oil |

D

| | |
|---|---|
| 2.00 | Iron oxide |
| 12.00 | Titanium dioxide |

### Preparation:

Heat phase A and phase B separately from one another to 80° C. Then mix phase B into phase A using a stirrer. Allow everything to cool to 40° C. and add phase C and phase D. Homogenize again.

The example can be repeated in each case with the polymers 1 and 3, 4 and 10 to 13 according to the invention. In each case, a liquid makeup with good properties is obtained.

### Example 20

### Oil-Free Makeup

A

| | |
|---|---|
| 0.35 | Veegum |
| 5.00 | Butylene glycol |
| 0.15 | Xanthan gum |

B

| | |
|---|---|
| 34.0 | Dist. water |
| q.s. | Preservative |
| 0.2 | Polysorbate-20 |
| 1.6 | Tetrahydroxypropylethylenediamine |

C

| | |
|---|---|
| 1.0 | Silicon dioxide |
| 2.0 | Nylon-12 |
| 4.15 | Mica |
| 6.0 | Titanium dioxide |
| 1.85 | Iron oxide |

D

| | |
|---|---|
| 4.0 | Stearic acid |
| 1.5 | Glyceryl stearate |
| 7.0 | Benzyl laurate |
| 5.0 | Isoeicosane |
| q.s. | Preservative |

E

| | |
|---|---|
| 0.5 | Panthenol |
| 0.1 | Imidazolidinylurea |
| 5.0 | Polymer from example 2 (solid) |

### Preparation:

Wet phase A with butylene glycol, add to phase B and mix well. Heat phase AB to 75° C. Pulverize phase C feed substances, add to phase AB and homogenize well. Mix feed substances of phase D, heat to 80° C. and add to phase ABC. Mix for some time until everything is homogeneous. Transfer everything to a vessel with propellor mixer. Mix the feed substances of phase E, add to phase ABCD and mix well.

The example can be repeated in each case with the polymers 1 and 3, 4 and 10 to 13 according to the invention. In each case, an oil-free makeup with good properties is obtained.

### Example 21

### Shimmering Gel

A

| | |
|---|---|
| 32.6 | Dist. water |
| 0.1 | Disodium EDTA |
| 25.0 | Natrosol (4% strength aqueous solution) |
| 0.3 | Preservative |

B

| | |
|---|---|
| 0.5 | Dist. water |
| 0.5 | Triethanolamine |

C

| | |
|---|---|
| 2.0 | Polymer from example 2 (solid) |
| ad 100 | Dist. water |
| 1.0 | Polyquaternium-46 (20% strength aqueous solution) |
| 5.0 | Iron oxide |

D

| | |
|---|---|
| 15.0 | Dist. water |
| 1.0 | D-Panthenol 50 P (panthenol and propylene glycol) |

### Preparation:

Using a propeller mixer, thoroughly mix the feed substances of phase A in the order given. Then add phase B to phase A. Stir slowly until everything is homogeneous.

Thoroughly homogenize phase C until the pigments are well distributed. Add phase C and phase D to phase AB and mix well.

The example can be repeated in each case with the polymers 1 and 3, 4 and 10 to 13 according to the invention. In each case, a shimmering gel with good properties is obtained.

### Example 22

### Sunscreen Gel

Phase A

| | |
|---|---|
| 1.00 | hydrogenated castor oil-PEG-40 |
| 8.00 | Octyl methoxycinnamate (Uvinul ® MC 80) |
| 5.00 | Octocrylene (Uvinul ® N 539) |
| 0.80 | Octyl Triazone (Uvinul ® T 150) |
| 2.00 | Butyl Methoxydibenzoylmethane (Uvinul ® BMBM) |
| 2.00 | Tocopheryl acetate |
| q.s. | Perfume oil |

Phase B

| | |
|---|---|
| 2.50 | Polymer from example 2 (solid) |
| ad 100 | Dist. water |
| 0.30 | Acrylate/C₁₀₋₃₀ alkyl acrylate copolymer |
| 0.20 | Carbomer |
| 5.00 | Glycerol |
| 0.20 | Disodium EDTA |
| q.s. | Preservative |
| 62.80 | Dist. water |

Phase C

| | |
|---|---|
| 0.20 | Sodium hydroxide |

### Preparation:

Mix the components of phase A. Allow phase B to swell and stir into phase A with homogenization. Neutralize with phase C and homogenize again.

The example can be repeated in each case with the polymers 1 and 3, 4 and 10 to 13 according to the invention. In each case, a sunscreen gel with good properties is obtained.

### Example 23

### Sunscreen emulsion with TiO₂ and ZnO₂

Phase A

| | |
|---|---|
| 6.00 | hydrogenated castor oil-PEG-7 |
| 2.00 | PEG-45/Dodecyl Glycol Copolymer |
| 3.00 | Isopropyl myristate |
| 8.00 | Jojoba oil (Buxus Chinensis) |
| 4.00 | Octyl Methoxycinnamate (Uvinul ® MC 80) |
| 2.00 | 4-Methylbenzylidenecamphor (Uvinul ® MBC 95) |
| 3.00 | Titanium dioxide, dimethicone |
| 1.00 | Dimethicone |
| 5.00 | Zinc oxide, dimethicone |

Phase B

| | |
|---|---|
| 2.0 | Polymer from example 2 (solid) |
| ad 100 | Dist. water |
| 0.20 | Disodium EDTA |
| 5.00 | Glycerol |
| q.s. | Preservative |
| 50.80 | Dist. water |

Phase C

| | |
|---|---|
| q.s. | Perfume oil |

### Preparation:

Heat phases A and B separately to about 85° C. Stir phase B into phase A and homogenize. Cool to about 40° C., add phase C and briefly homogenize again.

The example can be repeated in each case with the polymers 1 and 3, 4 and 10 to 13 according to the invention. In each case, a sunscreen emulsion with TiO₂ and ZnO₂ with good properties is obtained.

### Example 24

### Sunscreen Lotion

Phase A

| | |
|---|---|
| 6.00 | Octyl Methoxycinnamate (Uvinul ® MC 80) |
| 2.50 | 4-Methylbenzylidenecamphor (Uvinul ® MBC 95) |
| 1.00 | Octyl Triazone (Uvinul ® T 150) |
| 2.00 | Butyl Methoxydibenzoylmethane (Uvinul ® BMBM) |
| 2.00 | PVP/Hexadecene copolymer |
| 5.00 | PPG-3 Myristyl Ether |
| 0.50 | Dimethicone |
| 0.10 | BHT, ascorbyl palmitate, citric acid, glyceryl stearate |
| | propylene glycol |
| 2.00 | Cetyl alcohol |
| 2.00 | Potassium cetyl phosphate |

Phase B

| | |
|---|---|
| 0.50 | Polymer from example 2 (solid) |
| ad 100 | Dist. water |
| 5.00 | Propylene glycol |
| 0.20 | Disodium EDTA |
| q.s. | Preservative |
| 63.92 | Dist. water |

Phase C

| | |
|---|---|
| 5.00 | Mineral oil |
| 0.20 | Carbomer |

Phase D

| | |
|---|---|
| 0.08 | Sodium hydroxide |

Phase E

| | |
|---|---|
| q.s. | Perfume oil |

### Preparation:

Heat phases A and B separately to about 80° C. Stir phase B into phase A with homogenization, briefly after-homogenize. Prepare a slurry from phase C, stir into phase AB, neutralize with phase D and after-homogenize. Cool to about 40° C., add phase E, homogenize again.

The example can be repeated in each case with the polymers 1 and 3, 4 and 10 to 13 according to the invention. In each case, a sunscreen lotion with good properties is obtained.

### Example 25

### Peelable Face Mask

Phase A

| | |
|---|---|
| 57.10 | Dist. water |
| 6.00 | Polyvinyl alcohol |
| 5.00 | Propylene glycol |

Phase B

| | |
|---|---|
| 20.00 | Alcohol |
| 4.00 | PEG-32 |
| q.s | Perfume oil |

Phase C

| | |
|---|---|
| 5.00 | Polyquaternium-44 |
| 0.50 | Polymer from Example 2 (solid) |
| ad 100 | Dist. water |
| 0.20 | Allantoin |

### Preparation:

Heat phase A to at least 90° C. and stir until dissolved. Dissolve phase B at 50° C. and stir into phase A. At about 35° C., compensate for the loss of ethanol. Add phase C and stir in.

The example can be repeated in each case with the polymers 1 and 3, 4 and 10 to 13 according to the invention. In each case, a peelable face mask with good properties is obtained.

### Example 26

### Face Mask

Phase A

| | |
|---|---|
| 3.00 | Ceteareth-6 |
| 1.50 | Ceteareth-25 |
| 5.00 | Cetearyl alcohol |
| 6.00 | Cetearyl octanoate |
| 6.00 | Mineral oil |
| 0.20 | Bisabolol |
| 3.00 | Glyceryl stearate |

Phase B

| | |
|---|---|
| 2.00 | Propylene glycol |
| 5.00 | Panthenol |
| 2.50 | Polymer from Example 2 (solid) |
| ad 100 | Dist. water |
| q.s. | Preservative |
| 53.80 | Dist. water |

Phase C

| | |
|---|---|
| q.s. | Perfume oil |
| 0.50 | Tocopheryl acetate |

### Preparation:

Heat phases A and B separately to about 80° C. Stir phase B into phase A with homogenization, briefly after-homogenize. Cool to about 40° C., add phase C, homogenize again.

The example can be repeated in each case with the polymers 1 and 3, 4 and 10 to 13 according to the invention. In each case, a face mask with good properties is obtained.

### Example 27

### Body Lotion Foam

Phase A

| | |
|---|---|
| 1.50 | Ceteareth-25 |
| 1.50 | Ceteareth-6 |
| 4.00 | Cetearyl alcohol |
| 10.00 | Cetearyl octanoate |
| 1.00 | Dimethicone |

Phase B

| | |
|---|---|
| 0.50 | Polymer from Example 2 (solid) |
| ad 100 | Dist. water |
| 2.00 | Panthenol |
| 2.50 | Propylene glycol |
| q.s. | Preservative |
| 74.50 | Dist. water |

Phase C

| | |
|---|---|
| q.s. | Perfume oil |

### Preparation:

Heat phases A and B separately to about 80° C. Stir phase B into phase A and homogenize. Cool to about 40° C., add phase C and briefly homogenize again. Bottling: 90% active substance and 10% propane/butane at 3.5 bar (20° C.).

The example can be repeated in each case with the polymers 1 and 3, 4 and 10 to 13 according to the invention. In each case, a body lotion foam with good properties is obtained.

### Example 28

### Face Tonic for Dry and Sensitive Skin

Phase A

| | |
|---|---|
| 2.50 | hydrogenated castor oil-PEG-40 |
| q.s. | Perfume oil |
| 0.40 | Bisabolol |

Phase B

| | |
|---|---|
| 3.00 | Glycerol |
| 1.00 | Hydroxyethylcetyldimonium phosphate |
| 5.00 | Witch hazel (Hamamelis Virginiana) distillate |
| 0.50 | Panthenol |
| 0.1 | Polymer from Example 2 (solid) |
| ad 100 | Dist. water |
| q.s. | Preservative |
| 87.60 | Dist. water |

### Preparation:

Dissolve phase A to give a clear solution. Stir phase B into phase A.

The example can be repeated in each case with the polymers 1 and 3, 4 and 10 to 13 according to the invention. In each case, a face tonic for dry and sensitive skin with good properties is obtained.

### Example 29

### Face Washing Paste with Peeling Effect

Phase A

| | |
|---|---|
| 58.00 | Dist. water |
| 2.50 | Polymer from Example 2 (solid) |
| ad 100 | Dist. water |
| 1.50 | Carbomer |
| q.s. | Preservative |

Phase B

| | |
|---|---|
| q.s. | Perfume oil |
| 7.00 | Potassium Cocoyl Hydrolyzed Protein |
| 4.00 | Cocamidpropylbetaine |

Phase C

| | |
|---|---|
| 1.50 | Triethanolamine |

Phase D

| | |
|---|---|
| 13.00 | Polyethylene (Luwax ® A) |

### Preparation:

Allow phase A to swell. Dissolve phase B to give a clear solution. Stir phase B into phase A. Neutralize with phase C. Then stir in phase D.

The example can be repeated in each case with the polymers 1 and 3, 4 and 10 to 13 according to the invention. In each case, a face washing paste with peeling effect with good properties is obtained.

### Example 30

### Face Soap

Phase A

| | |
|---|---|
| 25.0 | Potassium cocoate |
| 20.0 | Disodium Cocoamphodiacetate |
| 2.0 | Lauramide DEA |
| 1.0 | Glycol stearate |
| 0.50 | Polymer from Example 2 (solid) |
| ad 100 | Dist. water |
| 50.0 | Dist. water |
| q.s. | Citric acid |

Phase B

| | |
|---|---|
| q.s. | Preservative |
| q.s. | Perfume oil |

### Preparation:

Heat phase A to 70° C. with stirring until everything is homogeneous. Adjust pH to 7.0-7.5 with citric acid, allow everything to cool to 50° C. and add phase B.

The example can be repeated in each case with the polymers 1 and 3, 4 and 10 to 13 according to the invention. In each case, a face soap with good properties is obtained.

### Example 31

### Face Cleansing Milk, O/W Type

Phase A

| | |
|---|---|
| 1.50 | Ceteareth-6 |
| 1.50 | Ceteareth-25 |
| 2.00 | Glyceryl stearate |
| 2.00 | Cetyl alcohol |
| 10.00 | Mineral oil |

Phase B

| | |
|---|---|
| 5.00 | Propylene glycol |
| q.s. | Preservative |
| 1.0 | Polymer from Example 2 (solid) |
| ad 100 | Dist. water |
| 62.30 | Dist. water |

Phase C

| | |
|---|---|
| 0.20 | Carbomer |
| 10.00 | Cetearyl octanoate |

Phase D

| | |
|---|---|
| 0.40 | Tetrahydroxypropylethylenediamine |

Phase E

| | |
|---|---|
| q.s. | Perfume oil |
| 0.10 | Bisabolol |

### Preparation:

Heat phases A and B separately to about 80° C. Stir phase B into phase A with homogenization, briefly after-homogenize. Prepare a slurry from phase C, stir into phase AB, neutralize with phase D and after-homogenize. Cool to about 40° C., add phase E, homogenize again.

The example can be repeated in each case with the polymers 1 and 3, 4 and 10 to 13 according to the invention. In each case, a face cleansing milk, O/W type with good properties is obtained.

### Example 32

### Peeling Cream, O/W Type

Phase A

| | |
|---|---|
| 3.00 | Ceteareth-6 |
| 1.50 | Ceteareth-25 |
| 3.00 | Glyceryl stearate |
| 5.00 | Cetearyl alcohol, sodium cetearyl sulfate |
| 6.00 | Cetearyl octanoate |
| 6.00 | Mineral oil |
| 0.20 | Bisabolol |

Phase B

| | |
|---|---|
| 2.00 | Propylene glycol |
| 0.10 | Disodium EDTA |
| 0.50 | Polymer from Example 2 (solid) |
| ad 100 | Dist. water |
| q.s. | Preservative |
| 59.70 | Dist. water |

Phase C

| | |
|---|---|
| 0.50 | Tocopheryl acetate |
| q.s. | Perfume oil |

Phase D

| | |
|---|---|
| 10.00 | Polyethylene |

### Preparation:

Heat phases A and B separately to about 80° C. Stir phase B into phase A and homogenize. Cool to about 40° C., add phase C and briefly homogenize again. Then stir in phase D.

The example can be repeated in each case with the polymers 1 and 3, 4 and 10 to 13 according to the invention. In each case, a peeling cream, O/W type with good properties is obtained.

### Example 33

### Shaving Foam

| | |
|---|---|
| 6.00 | Ceteareth-25 |
| 5.00 | Poloxamer 407 |
| 52.00 | Dist. water |
| 1.00 | Triethanolamine |
| 5.00 | Propylene glycol |
| 1.00 | Lanolin oil-PEG-75 |
| 1.0 | Polymer from Example 2 (solid) |
| ad 100 | Dist. water |
| q.s. | Preservative |
| q.s. | Perfume oil |
| 25.00 | Sodium laureth sulfate |

### Preparation:

Weigh everything together, then stir until dissolved. Bottling: 90 parts of active substance and 10 parts of 25:75 propane/butane mixture.

The example can be repeated in each case with the polymers 1 and 3, 4 and 10 to 13 according to the invention. In each case, a shaving foam with good properties is obtained.

### Example 34

### Aftershave Balsam

Phase A

| | |
|---|---|
| 0.25 | Acrylate/C₁₀₋₃₀ alkyl acrylate copolymer |
| 1.50 | Tocopheryl acetate |
| 0.20 | Bisabolol |
| 10.00 | Caprylic/Capric triglyceride |
| q.s. | Perfume oil |
| 1.00 | hydrogenated castor oil-PEG-40 |

Phase B

| | |
|---|---|
| 1.00 | Panthenol |
| 15.00 | Alcohol |
| 5.00 | Glycerol |
| 0.05 | Hydroxyethylcellulose |
| 0.50 | Polymer from Example 2 (solid) |
| ad 100 | Dist. water |
| 64.00 | Dist. water |

Phase C

| | |
|---|---|
| 0.08 | Sodium hydroxide |

### Preparation:

Mix the components of phase A. Stir phase B into phase A with homogenization, briefly after-homogenize. Neutralize with phase C and homogenize again.

The example can be repeated in each case with the polymers 1 and 3, 4 and 10 to 13 according to the invention. In each case, an aftershave balsam with good properties is obtained.

### Example 35

### Bodycare Cream

Phase A

| | |
|---|---|
| 2.00 | Ceteareth-6 |
| 2.00 | Ceteareth-25 |
| 2.00 | Cetearyl alcohol |
| 3.00 | Glyceryl stearate SE |
| 5.00 | Mineral oil |
| 4.00 | Jojoba oil (Buxus Chinensis) |
| 3.00 | Cetearyl octanoate |
| 1.00 | Dimethicone |
| 3.00 | Mineral oil, Lanolin alcohol |

Phase B

| | |
|---|---|
| 5.00 | Propylene glycol |
| 0.50 | Veegum |
| 1.00 | Panthenol |
| 1.70 | Polymer from Example 1 (solid) |
| ad 100 | Dist. water |
| 6.00 | Polyquaternium-44 (10% strength aqueous solution) |
| q.s. | Preservative |
| 54.00 | Dist. water |

Phase C

| | |
|---|---|
| q.s. | Perfume oil |

### Preparation:

Heat phases A and B separately to about 80° C. Homogenize phase B. Stir phase B into phase A with homogenization, briefly after-homogenize. Cool to about 40° C., add phase C and briefly homogenize again.

The example can be repeated in each case with the polymers 1 and 3, 4 and 10 to 13 according to the invention. In each case, a bodycare cream with good properties is obtained.

### Example 36

### Toothpaste

Phase A

| | |
|---|---|
| 34.79 | Dist. water |
| 0.50 | Polymer from Example 2 (solid) |
| ad 100 | Dist. water |
| 0.30 | Preservative |
| 20.00 | Glycerol |
| 0.76 | Sodium monofluorophosphate |

Phase B

| | |
|---|---|
| 1.20 | Sodium carboxymethylcellulose |

Phase C

| | |
|---|---|
| 0.80 | Aroma oil |
| 0.06 | Saccharin |
| 0.10 | Preservative |
| 0.05 | Bisabolol |
| 1.00 | Panthenol |
| 0.50 | Tocopheryl acetate |
| 2.80 | Silicon dioxide |
| 1.00 | Sodium lauryl sulfate |
| 7.90 | Dicalcium phosphate, anhydrous |
| 25.29 | Dicalcium phosphate dihydrate |
| 0.45 | Titanium dioxide |

### Preparation:

Dissolve phase A. Sprinkle phase B into phase A and dissolve. Add phase C and leave to stir under reduced pressure at RT for about 45 min.

The example can be repeated in each case with the polymers 1 and 3, 4 and 10 to 13 according to the invention. In each case, a toothpaste with good properties is obtained.

### Example 37

### Mouthwash

Phase A

| | |
|---|---|
| 2.00 | Aroma oil |
| 4.00 | hydrogenated castor oil-PEG-40 |
| 1.00 | Bisabolol |
| 30.00 | Alcohol |

Phase B

| | |
|---|---|
| 0.20 | Saccharin |
| 5.00 | Glycerol |
| q.s. | Preservative |
| 5.00 | Poloxamer 407 |
| 0.50 | Polymer from Example 2 (solid) |
| ad 100 | Dist. water |

### Preparation:

Dissolve phase A and phase B separately to give clear solutions. Stir phase B into phase A.

The example can be repeated in each case with the polymers 1 and 3, 4 and 10 to 13 according to the invention. In each case, a mouthwash with good properties is obtained.

### Example 38

### Denture Adhesive

Phase A

| | |
|---|---|
| 0.20 | Bisabolol |
| 1.00 | Betacarotene |
| q.s. | Aroma oil |
| 20.00 | Cetearyl octanoate |
| 5.00 | Silicon dioxide |
| 33.80 | Mineral oil |

Phase B

| | |
|---|---|
| 1.0 | Polymer from Example 2 (solid) |
| ad 100 | Dist. water |
| 35.00 | PVP (20% strength solution in water) |

### Preparation:

Thoroughly mix phase A. Stir phase B into phase A.

The example can be repeated in each case with the polymers 1 and 3, 4 and 10 to 13 according to the invention. In each case, a denture adhesive with good properties is obtained.

### Example 39

### Skincare Cream, O/W Type

Phase A

| | |
|---|---|
| 8.00 | Cetearyl alcohol |
| 2.00 | Ceteareth-6 |
| 2.00 | Ceteareth-25 |
| 10.00 | Mineral oil |
| 5.00 | Cetearyl octanoate |
| 5.00 | Dimethicone |

Phase B

| | |
|---|---|
| 0.50 | Polymer from Example 2 (solid) |
| ad 100 | Dist. water |
| 2.00 | Panthenol, Propylene glycol |
| q.s. | Preservative |

Phase C

| | |
|---|---|
| q.s. | Perfume oil |

### Preparation:

Heat phases A and B separately to about 80° C. Stir phase B into phase A with homogenization, briefly after-homogenize. Cool to about 40° C., add phase C, homogenize again.

The example can be repeated in each case with the polymers 1 and 3, 4 and 10 to 13 according to the invention. In each case, a skincare cream, O/W type with good properties is obtained.

### Example 40

### Skincare Cream, W/O Type

Phase A

| | |
|---|---|
| 6.00 | hydrogenated castor oil-PEG-7 |
| 8.00 | Cetearyl octanoate |
| 5.00 | Isopropyl myristate |
| 15.00 | Mineral oil |
| 2.00 | PEG-45/dodecyl glycol copolymer |
| 0.50 | Magnesium stearate |
| 0.50 | Aluminum stearate |

Phase B

| | |
|---|---|
| 3.00 | Glycerol |
| 0.60 | Polymer from Example 2 (solid) |
| ad 100 | Dist. water |
| 0.70 | Magnesium sulfate |
| 2.00 | Panthenol |
| q.s. | Preservative |

Phase C

| | |
|---|---|
| 1.00 | Tocopherol |
| 5.00 | Tocopheryl acetate |
| q.s. | Perfume oil |

### Preparation:

Heat phases A and B separately to about 80° C. Stir phase B into phase A and homogenize. Cool to about 40° C., add phase C and briefly homogenize again.

The example can be repeated in each case with the polymers 1 and 3, 4 and 10 to 13 according to the invention. In each case, a skincare cream, W/O type with good properties is obtained.

### Example 41

### Lipcare Cream

Phase A

| | |
|---|---|
| 10.00 | Cetearyl octanoate |
| 5.00 | Polybutene |

Phase B

| | |
|---|---|
| 0.10 | Carbomer |

Phase C

| | |
|---|---|
| 2.00 | Ceteareth-6 |
| 2.00 | Ceteareth-25 |
| 2.00 | Glyceryl stearate |
| 2.00 | Cetyl alcohol |
| 1.00 | Dimethicone |
| 1.00 | Benzophenone-3 |
| 0.20 | Bisabolol |
| 6.00 | Mineral oil |

Phase D

| | |
|---|---|
| 1.50 | Polymer from Example 2 (solid) |
| ad 100 | Dist. water |
| 3.00 | Panthenol |
| 3.00 | Propylene glycol |
| q.s. | Preservative |

Phase E

| | |
|---|---|
| 0.10 | Triethanolamine |

Phase F

| | |
|---|---|
| 0.50 | Tocopheryl acetate |
| 0.10 | Tocopherol |
| q.s. | Perfume oil |

### Preparation:

Dissolve phase A to give a clear solution. Add phase B and homogenize. Add phase C and melt at 80° C. Heat phase D to 80° C. Add phase D to phase ABC and homogenize. Cool to about 40° C., add phase E and phase F, homogenize again.

The example can be repeated in each case with the polymers 1 and 3, 4 and 10 to 13 according to the invention. In each case, a lipcare cream with good properties is obtained.

### Example 42

### Shower Gel

| | |
|---|---|
| 50.00 | Sodium Laureth Sulfate, Magnesium Laureth Sulfate, |
| | Sodium Laureth-8 Sulfate, Magnesium Laureth-8 |
| 1.00 | Cocoamide DEA |
| 0.8 | Polymer from Example 2 (solid) |
| ad 100 | Dist. water |
| 2.00 | Sodium Laureth Sulfate, Glycol Distearate, Cocamide MEA, |
| | Laureth-10 |
| q.s. | Preservative |
| q.s. | Perfume oil |
| 2.00 | Sodium chloride |

### Preparation:

Weigh in all of the components together and stir until dissolved.

The example can be repeated in each case with the polymers 1 and 3, 4 and 10 to 13 according to the invention. In each case, a shower gel with good properties is obtained.

### Example 43

### Shower Gel

| | |
|---|---|
| 30.00 | Sodium Laureth Sulfate |
| 6.00 | Sodium Cocoamphodiacetate |
| 6.00 | Cocamidopropylbetaine |
| 3.00 | Sodium Laureth Sulfate, Glycol Distearate, Cocamide MEA, |
| | Laureth-10 |
| 7.70 | Polyquaternium-44 |
| 0.2 | Polymer from Example 2 (solid) |
| ad 100 | Dist. water |
| 1.00 | Panthenol |
| q.s. | Preservative |
| q.s. | Perfume oil |
| q.s. | Citric acid |
| 0.50 | Sodium chloride |

### Preparation:

Weigh in the components of phase A and dissolve. Adjust the pH to 6 to 7.

The example can be repeated in each case with the polymers 1 and 3, 4 and 10 to 13 according to the invention. In each case, a shower gel with good properties is obtained.

### Example 44

### Clear Shower Gel

| | |
|---|---|
| 40.00 | Sodium Laureth Sulfate |
| 5.00 | Decyl glucoside |
| 5.00 | Cocamidopropylbetaine |
| 0.50 | Polyquaternium-10 |
| 2.00 | Polymer from Example 2 (solid) |
| ad 100 | Dist. water |
| 1.00 | Panthenol |
| q.s. | Perfume oil |
| q.s. | Preservative |
| q.s. | Citric acid |
| 2.00 | Sodium chloride |

### Preparation:

Weigh in the components of phase A and dissolve to give a clear solution.

The example can be repeated in each case with the polymers 1 and 3, 4 and 10 to 13 according to the invention. In each case, a clear shower gel with good properties is obtained.

### Example 45

### Shower Bath

A

| | |
|---|---|
| 40.00 | Sodium Laureth Sulfate |
| 5.00 | Sodium C₁₂₋₁₅ Pareth-15 Sulfonate |
| 5.00 | Decyl glucoside |
| q.s. | Perfume oil |
| 0.10 | Phytantriol |

B

| | |
|---|---|
| 0.1 | Guar hydroxypropyltrimonium chloride |
| 2.00 | Polymer from Example 2 (solid) |
| ad 100 | Dist. water |
| 1.00 | Panthenol |
| q.s. | Preservative |
| 1.00 | Laureth-3 |
| q.s. | Citric acid |
| 2.00 | Sodium chloride |

### Preparation:

Mix the components of phase A. Add the components of phase B one after the other and mix. Adjust the pH to 6 to 7.

The example can be repeated in each case with the polymers 1 and 3, 4 and 10 to 13 according to the invention. In each case, a shower bath with good properties is obtained.

### Example 46

### Liquid Soap

A

| | |
|---|---|
| 43.26 | Dist. water |
| 0.34 | Aminomethylpropanol |
| 3.40 | Poly(ethyl acrylate/methacrylic acid) (Luviflex ® Soft, BASF) |

B

| | |
|---|---|
| 40.00 | Sodium Laureth Sulfate |
| 10.00 | Cocamidopropylbetaine |
| 0.2 | Polymer from Example 2 (solid) |
| ad 100 | Dist. water |
| q.s. | Perfume oil |
| q.s. | Preservative |
| 2.00 | Sodium chloride |

### Preparation:

Weigh in the components of phase A and dissolve to give a clear solution. Add the components of phase B one after the other and mix.

The example can be repeated in each case with the polymers 1 and 3, 4 and 10 to 13 according to the invention. In each case, a liquid soap with good properties is obtained.

### Example 47

### Liquid Foot Bath

A

| | |
|---|---|
| 1.00 | Nonoxynol-14 |
| 0.10 | Bisabolol |
| 1.00 | Pine oil (Pinus Sylvestris) |

B

| | |
|---|---|
| 5.00 | PEG-8 |
| 1.50 | Polymer from Example 2 (solid) |
| ad 100 | Dist. water |
| 0.50 | Triclosan |
| 30.00 | Sodium Laureth Sulfate |
| 3.00 | Polyquaternium-16 |
| q.s. | C.I. 19 140 + C.I. 42 051 |

### Preparation:

### Solubilize Phase A. Mix Phase B.

The example can be repeated in each case with the polymers 1 and 3, 4 and 10 to 13 according to the invention. In each case, a liquid foot bath with good properties is obtained.

### Example 48

### Freshening Gel

A

| | |
|---|---|
| 0.60 | Carbomer |
| 45.40 | Dist. water |

B

| | |
|---|---|
| 0.50 | Bisabolol |
| 0.50 | Farnesol |
| q.s. | Perfume oil |
| 5.00 | PEG-40 Hydrogenated Castor Oil |
| 0.50 | Polymer from Example 2 (solid) |
| ad 100 | Dist. water |
| 1.00 | Tetrahydroxypropylethylenediamine |
| 1.50 | Menthol |
| 43.00 | Alcohol |
| q.s. | C.I. 74 180, Direct Blue 86 |

### Preparation:

Allow phase A to swell. Dissolve phase B. Stir phase B into phase A.

The example can be repeated in each case with the polymers 1 and 3, 4 and 10 to 13 according to the invention. In each case, a freshening gel with good properties is obtained.

### Example 49

### Roll-On Antiperspirant

A

| | |
|---|---|
| 0.40 | Hydroxyethylcellulose |
| 50.00 | Dist. water |

B

| | |
|---|---|
| 25.00 | Alcohol |
| 0.10 | Bisabolol |
| 0.30 | Farnesol |
| 2.00 | PEG-40 Hydrogenated Castor Oil |
| q.s. | Perfume oil |

C

| | |
|---|---|
| 5.00 | Aluminum chlorohydrate |
| 3.00 | Propylene glycol |
| 3.00 | Dimethicone copolyol |
| 3.00 | Polyquaternium-16 |
| 1.50 | Polymer from Example 2 (solid) |
| ad 100 | Dist. water |

### Preparation:

Allow phase A to swell. Dissolve phase B and C separately. Stir phase A and B into phase C.

The example can be repeated in each case with the polymers 1 and 3, 4 and 10 to 13 according to the invention. In each case, a roll-on antiperspirant with good properties is obtained.

### Example 50

### Transparent Deodorant Stick

| | |
|---|---|
| 5.00 | Sodium stearate |
| 0.50 | Triclosan |
| 3.00 | Ceteareth-25 |
| 20.00 | Glycerol |
| 0.50 | Polymer from Example 2 (solid) |
| ad 100 | Dist. water |
| q.s. | Perfume oil |
| 60.00 | Propylene glycol |
| 0.20 | Bisabolol |

### Preparation:

Weigh phase A together, melt and homogenize. Then pour into the mold.

The example can be repeated in each case with the polymers 1 and 3, 4 and 10 to 13 according to the invention. In each case, a transparent deodorant stick with good properties is obtained.

### Example 51

### Water-Soluble Bath Oil

| | |
|---|---|
| 15.00 | Cetearyl octanoate |
| 15.00 | Caprylic/Capric triglyceride |
| 1.00 | Panthenol, Propylene glycol |
| 0.10 | Bisabolol |
| 2.00 | Tocopheryl acetate |
| 2.00 | Retinyl palmitate |
| 0.10 | Tocopherol |
| 37.00 | PEG-7 glyceryl cocoate |
| 0.4 | Polymer from Example 2 (solid) |
| ad 100 | Dist. water |
| q.s. | Perfume oil |
| 23.60 | PEG-40 Hydrogenated Castor Oil |

### Preparation:

Mix and stir until everything has dissolved to give a clear solution.

The example can be repeated in each case with the polymers 1 and 3, 4 and 10 to 13 according to the invention. In each case, a water-soluble bath oil with good properties is obtained.

### Example 52

### Daycare Aerosol

A

| | |
|---|---|
| 4.00 | Ethylhexyl methoxycinnamate |
| 1.50 | Octocrylene |
| 9.00 | Caprylic/Capric triglyceride |
| 5.00 | Simmondsia Chinensis (Jojoba) Seed Oil |
| 1.50 | Cyclomethicone |
| 3.00 | Hydrogenated Cocoglycerides |
| 1.00 | PVP/Hexadecene copolymer |
| 1.00 | Ceteareth-6, stearyl alcohol |

B

| | |
|---|---|
| 5.00 | Zinc oxide |

C

| | |
|---|---|
| 2.00 | Ceteareth-25 |
| 1.20 | Panthenol |
| 0.20 | Sodium Ascorbyl Phosphate |
| 0.30 | Imidazolidinylurea |
| 0.10 | Disodium EDTA |
| 1.50 | Polymer from Example 2 (solid) |
| ad 100 | Dist. water |

D

| | |
|---|---|
| 0.50 | Tocopheryl acetate |
| 0.20 | Bisabolol |
| 0.33 | Caprylic/Capric triglyceride, Retinol |
| q.s. | Perfume oil |

### Preparation:

Heat phase A to 80° C. Dissolve phase A to give a clear solution. Work in phase B and homogenize. Add phase C, heat to 80° C., melt and homogenize. Cool with stirring to about 40° C., add phase D and briefly homogenize. Bottle 90% active ingredient solution: 10% propane/butane at 3.5 bar (20° C.).

The example can be repeated in each case with the polymers 1 and 3, 4 and 10 to 13 according to the invention. In each case, a daycare aerosol with good properties is obtained.

### Example 53

### Moisturizing Cream

A

| | |
|---|---|
| 3.00 | Vitis Vinifera (Grape) Seed Oil |
| 1.00 | Cyclopentasiloxane, cyclohexasiloxane |
| 1.50 | Cyclomethicone |
| 2.00 | Soybean (Glycine Soya) Oil |
| 2.00 | Ethylhexyl methoxycinnamate |
| 1.00 | Uvinul ® A Plus |
| 1.00 | Hydrogenated Lecithin |
| 1.00 | Cholesterol |
| 2.00 | PEG-40 Hydrogenated Castor Oil |
| 5.00 | Cetearyl octanoate |
| 5.00 | Caprylic/Capric triglyceride |

B

| | |
|---|---|
| 3.00 | Caprylic/Capric triglyceride, Acrylate copolymer |

C

| | |
|---|---|
| 2.50 | Polymer from Example 2 (solid) |
| ad 100 | Dist. water |
| 0.50 | Cocotrimonium methosulfate |
| 2.00 | Panthenol, Propylene glycol |
| 3.00 | Glycerol |
| 0.10 | Disodium EDTA |

D

| | |
|---|---|
| 0.30 | Perfume oil |
| 0.30 | DMDM Hydantoin |
| 1.00 | Tocopheryl acetate |
| 2.00 | Tocopherol |

### Preparation:

Heat phase A to 80° C. Stir phase B into phase A. Heat phase C to about 80° C. and stir into phase A+B with homogenization. Cool to about 40° C. with stirring. Add phase D and briefly homogenize.

The example can be repeated in each case with the polymers 1 and 3, 4 and 10 to 13 according to the invention. In each case, a moisturizing cream with good properties is obtained.

### Example 54

### Aerosol Hair Foam

A

| | |
|---|---|
| 2.00 | Cocotrimonium methosulfate |
| 0.20 | Perfume oil |

B

| | |
|---|---|
| 1.60 | Polymer from Example 2 (solid) |
| ad 100 | Dist. water |
| 0.50 | Poly(Ethylacrylate/methacrylic acid) (Luviflex ® Soft) |
| 0.10 | Aminomethylpropanol |
| 0.20 | Ceteareth-25 |
| 0.20 | Trimethylsilylamodimethicone, Trideceth-10, |
| | Cetrimonium Chloride |
| 0.10 | PEG-25 PABA |
| 0.20 | Hydroxyethylcellulose |
| 0.20 | PEG-8 |
| 0.20 | Panthenol |
| 15.00 | Alcohol |

C

| | |
|---|---|
| 10.00 | Propane/butane 3.5 bar (20° C.) |

### Preparation:

Mix phases A and B and bottle with propellant gas.

The example can be repeated in each case with the polymers 1 and 3, 4 and 10 to 13 according to the invention. In each case, an aerosol hair foam with good properties is obtained.

### Example 55

### Pump Mousse

A

| | |
|---|---|
| 2.00 | Cocotrimonium methosulfate |
| q.s. | Perfume oil |

C

| | |
|---|---|
| 7.00 | Polyquaternium-46 (10% strength aqueous solution) |
| 2.50 | Polymer from Example 2 (solid) |
| ad 100 | Dist. water |
| 0.50 | PEG-8 |
| 1.00 | Panthenol |
| q.s. | Preservative |
| 0.20 | PEG-25 PABA (ethoxylated p-aminobenzoic acid) |

### Preparation:

Mix the components of phase A. Add the components of phase B one after the other and dissolve to give a clear solution.

The example can be repeated in each case with the polymers 1 and 3, 4 and 10 to 13 according to the invention. In each case, a pump mousse with good properties is obtained.

### Example 56

### Aerosol Foam

| | |
|---|---|
| 3.0 | Polymer from Example 2 (solid) |
| ad 100 | Dist. water |
| 5.00 | PVP/VA-Copolymer (40% strength aqueous solution) |
| 0.50 | Hydroxyethylcetyldimonium phosphate |
| 0.20 | Ceteareth-25 |
| 0.40 | Perfume oil PC 910.781/Cremophor |
| q.s. | Preservative |
| 10.00 | Propane/butane 3.5 bar (20° C.) |

### Preparation:

Weigh everything together, stir until dissolved, then bottle.

The example can be repeated in each case with the polymers 1 and 3, 4 and 10 to 13 according to the invention. In each case, an aerosol foam with good properties is obtained.

### Example 57

### Color Styling Mousse

A

| | |
|---|---|
| 2.00 | Cocotrimonium methosulfate |
| q.s. | Perfume oil |

B

| | |
|---|---|
| 6.50 | Polymer from Example 2 (solid) |
| ad 100 | Dist. water |
| 0.50 | Acrylate copolymer (Luvimer ® 100 P, BASF) |
| 0.10 | Aminomethylpropanol |
| 0.20 | Ceteareth-25 |
| 0.20 | Panthenol |
| 0.20 | Hydroxyethylcellulose |
| 10.00 | Alcohol |
| 0.08 | C.I. 12245, Basic Red 76 |
| 0.05 | C.I. 42510, Basic Violet 14 |

C

| | |
|---|---|
| 10.00 | Propane/butane 3.5 bar (20° C.) |

### Preparation:

Weigh everything together, stir until dissolved, then bottle. Only suitable for dark blonde and brown hair!

The example can be repeated in each case with the polymers 1 and 3, 4 and 10 to 13 according to the invention. In each case, a color styling mousse with good properties is obtained.

### Example 58

### Pump Hair Foam

A

| | |
|---|---|
| 1.50 | Cocotrimonium methosulfate |
| q.s. | Perfume oil |

B

| | |
|---|---|
| 2.00 | Polymer from Example 2 (solid) |
| ad 100 | Dist. water |

C

| | |
|---|---|
| 0.46 | Aminomethylpropanol |
| 4.00 | PEG/PPG-25/25 Dimethicone/Acrylate copolymer |
| q.s. | Preservative |

### Preparation:

Mix phase A. Stir phase B into phase A. Add phase C and stir until dissolved.

The example can be repeated in each case with the polymers 1 and 3, 4 and 10 to 13 according to the invention. In each case, a pump hair foam with good properties is obtained.

### Example 59

### Aquawax

| | |
|---|---|
| 10 | Polymer from Example 2 (solid) |
| ad 100 | Dist. water |
| q.s. | Perfume oil |
| q.s. | hydrogenated castor oil-PEG-40 |
| 0.10 | Diethyl phthalate |
| 0.10 | Cetearyl ethylhexanoate |
| 0.10 | PEG-7 Glyceryl Cocoate |
| 0.10 | Preservative |
| 2.00 | Caprylic/Capric triglyceride, Acrylate copolymer |

### Preparation:

Mix everything and homogenize. After-stir for 15 minutes.

The example can be repeated in each case with the polymers 1 and 3, 4 and 10 to 13 according to the invention. In each case, an aquawax with good properties is obtained.

### Example 60

### Rinse-Off Conditioner and Repair Treatment

A

| | |
|---|---|
| 0.20 | Cetearyl octanoate |
| 0.10 | Phytantriol |
| 2.00 | hydrogenated castor oil-PEG-40 |

B

| | |
|---|---|
| q.s. | Perfume oil |
| 2.00 | Cocotrimonium methosulfate |

C

| | |
|---|---|
| ad 100 | Dist. water |

D

| | |
|---|---|
| 2.00 | Polyquaternium-16 (20% strength aqueous solution) |
| 1.0 | Polymer from Example 2 (solid) |
| 1.00 | Dimethicone copolyol |
| q.s. | Preservative |
| 10.00 | Alcohol |
| q.s. | Citric acid |

### Preparation:

Mix phases A and B separately. Stir phase C into phase B.

The example can be repeated in each case with the polymers 1 and 3, 4 and 10 to 13 according to the invention. In each case, a rinse-off conditioner and repair treatment with good properties is obtained.

### Example 61

### Hair Treatment

A

| | |
|---|---|
| 2.00 | Ceteareth-6, Stearyl alcohol |
| 1.00 | Ceteareth-25 |
| 6.00 | Cetearyl alcohol |
| 6.00 | Cetearyl octanoate |
| 0.30 | Phytantriol |

B

| | |
|---|---|
| 1.0 | Polymer from Example 2 (solid) |
| ad 100 | Dist. water |
| 0.70 | Guar hydroxypropyltrimonium chloride |
| 5.00 | Propylene glycol |
| 2.00 | Panthenol |
| 0.30 | Imidazolidinylurea |

C

| | |
|---|---|
| 2.00 | Cosi Silk Soluble |
| 0.20 | Perfume |
| 0.50 | Phenoxyethanol |

### Preparation:

Heat phases A and B separately to about 80° C. Homogenize phase B.

The example can be repeated in each case with the polymers 1 and 3, 4 and 10 to 13 according to the invention. In each case, a hair treatment with good properties is obtained.

### Example 62

### Hair Cocktail

A

| | |
|---|---|
| 0.40 | Acrylates/C₁₀₋₃₀ alkyl acrylate crosspolymer |
| 2.00 | Dimethicone |
| 3.00 | Cyclomethicone, dimethiconol |
| 2.00 | Phenyltrimethicone |
| 2.00 | Amodimethicone, Cetrimonium Chloride, Trideceth-10 |
| 0.50 | Dimethicone copolyol |
| 1.00 | Macadamia nut oil (Ternifolia) |
| 0.50 | Tocopheryl acetate |
| 1.00 | PEG-40 Hydrogenated Castor Oil |
| q.s. | Perfume oil |

B

| | |
|---|---|
| 0.3 | Polymer from Example 2 (solid) |
| ad 100 | Dist. water |
| 0.46 | Aminomethylpropanol |
| 4.00 | PEG/PPG-25/25 Dimethicone/Acrylate copolymer |

### Preparation:

Mix the components of phase A. Dissolve phase B. Stir phase B into phase A with homogenization.

The example can be repeated in each case with the polymers 1 and 3, 4 and 10 to 13 according to the invention. In each case, a hair cocktail with good properties is obtained.

### Example 63

### Permanent Wave

### Waving Solution

A

| | |
|---|---|
| 0.20 | Cocamidopropylbetaine |
| 0.20 | Polysorbate 20 |
| 1.55 | Polymer from Example 2 (solid) |
| ad 100 | Dist. water |
| 0.20 | Disodium EDTA |
| 0.20 | Hydroxyethylcellulose |

B

| | |
|---|---|
| 8.00 | Thioglycolic acid |

C

| | |
|---|---|
| 11.00 | Ammonium hydroxide |

D

| | |
|---|---|
| 5.00 | Ammonium carbonate |

### Preparation:

Weigh in the components of phase A and dissolve to give a clear solution. Stir phase B into phase A.

The example can be repeated in each case with the polymers 1 and 3, 4 and 10 to 13 according to the invention. In each case, a waving solution with good properties is obtained.

### Example 64

### Neutralizer

A

| | |
|---|---|
| 1.00 | PEG-40 Hydrogenated Castor Oil |
| 0.20 | Perfume oil |
| ad 100 | Dist. water |

B

| | |
|---|---|
| 0.20 | Cocamidopropylbetaine |
| 0.20 | Ceteareth-25 |
| 2.5 | Polymer from Example 2 (solid) |
| q.s. | Preservative |

C

| | |
|---|---|
| 2.30 | Hydrogen peroxide |

D

| | |
|---|---|
| q.s. | Phosphoric acid |

### Preparation:

Solubilize phase A. Add the components of phase B one after the other and dissolve to give a clear solution.

The example can be repeated in each case with the polymers 1 and 3, 4 and 10 to 13 according to the invention. In each case, a neutralizer with good properties is obtained.

### Example 65

### Dark Brown Permanent Hair Color (Oxidation Hair Color)

A

| | |
|---|---|
| 0.20 | Sodium sulfite |
| 0.05 | Disodium EDTA |
| 0.20 | p-Phenylenediamine |
| 0.30 | Resorcinol |
| 0.20 | 4-Amino-2-hydroxytoluene |
| 0.10 | m-Aminophenol |
| 1.50 | Oleyl alcohol |
| 4.50 | Propylene glycol |
| 2.30 | Sodium C₁₂₋₁₅ Pareth-15 Sulfonate |
| 20.00 | Oleic acid |
| ad 100 | Dist. water |

B

| | |
|---|---|
| 1.0 | Polymer from Example 2 (solid) |
| 13.70 | Ammonium hydroxide |
| 6.00 | isopropanol |
| q.s. | Perfume |

### Preparation:

Solubilize phase A. Add the components of phase B one after the other and mix.

The example can be repeated in each case with the polymers 1 and 3, 4 and 10 to 13 according to the invention. In each case, a dark brown permanent hair color (oxidation hair color) with good properties is obtained.

### Example 66

### Developer Emulsion (pH 3-4)

| | |
|---|---|
| 3.00 | Hexadecyl alcohol |
| 1.0 | Polymer from Example 2 (solid) |
| ad 100 | Dist. water |
| 1.00 | Ceteareth-20 |
| 1.00 | Sodium C₁₂₋₁₅ Pareth-15 Sulfonate |
| 6.00 | Hydrogen peroxide |
| 0.50 | Phosphoric acid |
| 0.01 | Acetanilide |

### Preparation:

Add the components one after the other and mix.

The example can be repeated in each case with the polymers 1 and 3, 4 and 10 to 13 according to the invention. In each case, a developer emulsion (pH 3-4) with good properties is obtained.

### Example 67

### Pale Brown Semipermanent Hair Color

| | |
|---|---|
| 10.00 | Cocodiethanolamide |
| 4.00 | Sodium dodecylbenzylsulfonate, 50% strength |
| 1.0 | Polymer from Example 2 (solid) |
| ad 100 | Dist. water |
| 6.00 | C₉₋₁₁ Pareth-3 |
| 2.50 | Sodium lauryl sulfate |
| 0.40 | 2-Nitro-p-phenylenediamine |
| 0.20 | HC Red No. 3 |
| 0.20 | HC Yellow No. 2 |

### Preparation:

Add the components one after the other and mix.

The example can be repeated in each case with the polymers 1 and 3, 4 and 10 to 13 according to the invention. In each case, a pale brown semipermanent hair color with good properties is obtained.

### Example 68

### Shampoo

| | |
|---|---|
| 30.00 | Sodium Laureth Sulfate |
| 6.00 | Sodium Cocoamphoacetate |
| 6.00 | Cocamidopropylbetaine |
| 3.00 | Sodium Laureth Sulfate, Glycol Distearate, Cocamide |
| | MEA, Laureth-10 |
| 1.0 | Polymer from Example 2 (solid) |
| 2.00 | Dimethicone |
| q.s. | Perfume |
| q.s. | Preservative |
| q.s. | Citric acid |
| 1.00 | Sodium chloride |
| ad 100 | Dist. water |

### Preparation:

Weigh in and dissolve the components. Adjust pH to 6 to 7.

The example can be repeated in each case with the polymers 1 and 3, 4 and 10 to 13 according to the invention. In each case, a shampoo with good properties is obtained.

### Example 69

### Shampoo

| | |
|---|---|
| 30.00 | Sodium Laureth Sulfate |
| 6.00 | Sodium Cocoamphoacetate |
| 6.00 | Cocamidopropylbetaine |
| 3.00 | Sodium Laureth Sulfate, Glycol |
| | Distearate, Cocamide MEA, Laureth-10 |
| 1.0 | Polymer from Example 2 (solid) |
| 2.00 | Amodimethicone |
| q.s. | Perfume |
| q.s. | Preservative |
| q.s. | Citric acid |
| 1.00 | Sodium chloride |
| ad 100 | Dist. water |

### Preparation:

Weigh in and dissolve the components. Adjust pH to 6 to 7.

The example can be repeated in each case with the polymers 1 and 3, 4 and 10 to 13 according to the invention. In each case, a shampoo with good properties is obtained.

### Example 70

### Shampoo

| | |
|---|---|
| 40.00 | Sodium Laureth Sulfate |
| 10.00 | Cocamidopropylbetaine |
| 3.00 | Sodium Laureth Sulfate, Glycol |
| | Distearate, Cocamide MEA, Laureth-10 |
| 1.0 | Polymer from Example 2 (solid) |
| 2.00 | Dow Corning 3052 |
| q.s. | Perfume |
| q.s. | Preservative |
| q.s. | Citric acid |
| 2.00 | Cocamido DEA |
| ad 100 | Dist. water |

### Preparation:

Weigh in and dissolve the components. Adjust the pH to 6 to 7.

The example can be repeated in each case with the polymers 1 and 3, 4 and 10 to 13 according to the invention. In each case, a shampoo with good properties is obtained.

### Example 71

### Antidandruff Shampoo

| | |
|---|---|
| 40.00 | Sodium Laureth Sulfate |
| 10.00 | Cocamidopropylbetaine |
| 10.00 | Disodium Laureth Sulfosuccinate |
| 2.50 | Sodium Laureth Sulfate, Glycol Distearate, |
| | Cocamide MEA, Laureth-10 |
| 1.0 | Polymer from Example 2 (solid) |
| 0.50 | Climbazole |
| q.s. | Perfume |
| q.s. | Preservative |
| 0.50 | Sodium chloride |
| ad 100 | Dist. water |

### Preparation:

Weigh in and dissolve the components. Adjust the pH to 6 to 7.

The example can be repeated in each case with the polymers 1 and 3, 4 and 10 to 13 according to the invention. In each case, an antidandruff shampoo with good properties is obtained.

### Example 72

### Shampoo

| | |
|---|---|
| 25.00 | Sodium Laureth Sulfate |
| 5.00 | Cocamidopropylbetaine |
| 2.50 | Sodium Laureth Sulfate, Glycol Distearate, |
| | Cocamide MEA, Laureth-10 |
| 1.0 | Polymer from Example 2 (solid) |
| q.s. | Perfume |
| q.s. | Preservative |
| 2.00 | Cocamido DEA |
| ad 100 | Dist. water |

### Preparation:

Weigh in and dissolve the components. Adjust the pH to 6 to 7.

The example can be repeated in each case with the polymers 1 and 3, 4 and 10 to 13 according to the invention. In each case, a shampoo with good properties is obtained.

### Example 73

### Shampoo

| | |
|---|---|
| 20.00 | Ammonium Laureth Sulfate |
| 15.00 | Ammonium Lauryl Sulfate |
| 5.00 | Cocamidopropylbetaine |
| 2.50 | Sodium Laureth Sulfate, Glycol Distearate, Cocamide |
| | MEA, Laureth-10 |
| 1.0 | Polymer from Example 2 (solid) |
| q.s. | Perfume |
| q.s. | Preservative |
| 0.50 | Sodium chloride |
| ad 100 | Dist. water |

### Preparation:

Weigh in and dissolve the components. Adjust the pH to 6 to 7.

The example can be repeated in each case with the polymers 1 and 3, 4 and 10 to 13 according to the invention. In each case, a shampoo with good properties is obtained.

### Example 74

### Clear Shower Gel

| | |
|---|---|
| 40.00 | Sodium Laureth Sulfate |
| 5.00 | Decyl glucoside |
| 5.00 | Cocamidopropylbetaine |
| 1.0 | Polymer from Example 2 (solid) |
| 1.00 | Panthenol |
| q.s. | Perfume |
| q.s. | Preservative |
| q.s. | Citric acid |
| 2.00 | Sodium chloride |
| ad 100 | Dist. water |

### Preparation:

Weigh in and dissolve the components. Adjust the pH to 6 to 7.

The example can be repeated in each case with the polymers 1 and 3, 4 and 10 to 13 according to the invention. In each case, a clear shower gel with good properties is obtained.

### Example 75

### Shampoo

| | |
|---|---|
| 12.00 | Sodium Laureth Sulfate |
| 1.50 | Decyl glucoside |
| 2.50 | Cocamidopropylbetaine |
| 5.00 | Cocoglucoside Glyceryl Oleate |
| 2.00 | Sodium Laureth Sulfate, Glycol Distearate, |
| | Cocamide MEA, Laureth-10 |
| 1.0 | Polymer from Example 2 (solid) |
| q.s. | Preservative |
| q.s. | Sunset Yellow C.I. 15 985 |
| q.s. | Perfume |
| 1.00 | Sodium chloride |
| ad 100 | Dist. water |

### Preparation:

Weigh in and dissolve the components. Adjust the pH to 6 to 7.

The example can be repeated in each case with the polymers 1 and 3, 4 and 10 to 13 according to the invention. In each case, a shampoo with good properties is obtained.

### Example 76

### Shampoo

A

| | |
|---|---|
| 40.00 | Sodium Laureth Sulfate |
| 5.00 | Sodium C₁₂₋₁₅ Pareth-15 Sulfonate |
| 5.00 | Decyl glucoside |
| q.s. | Perfume |
| 0.10 | Phytantriol |

B

| | |
|---|---|
| 1.0 | Polymer from Example 2 (solid) |
| ad 100 | Dist. water |
| 1.00 | Panthenol |
| q.s. | Preservative |
| 1.00 | Laureth-3 |
| q.s. | Citric acid |
| 2.00 | Sodium chloride |

### Preparation:

Weigh in and dissolve the components of phase A. Adjust the pH to 6 to 7. Add phase B and mix.

The example can be repeated in each case with the polymers 1 and 3, 4 and 10 to 13 according to the invention. In each case, a shampoo with good properties is obtained.

1. A cosmetic preparation comprising at least one polymer A which comprises, in copolymerized form,
a) 40-89.5% by weight of at least one ester of (meth)acrylic acid,
b) 10-49% by weight of at least one olefinically unsaturated, anionogenic or anionic compound,
c) 0.5-10% by weight of at least one compound chosen from
c1) polyesters comprising at least two free-radically polymerizable, olefinically unsaturated double bonds and
c2) polyethers comprising at least two free-radically polymerizable, olefinically unsaturated double bonds,
d) 0-30% by weight of at least one olefinically unsaturated compound
with the proviso that the amounts of components a) to d) add up to 100% by weight.

2. The cosmetic preparation according to claim 1, where component a) is chosen from the group consisting of methyl (meth)acrylate, ethyl (meth)acrylate, n-propyl (meth)acrylate, isopropyl (meth)acrylate, n-butyl (meth)acrylate, tert-butyl (meth)acrylate, isobutyl (meth)acrylate, sec-butyl (meth)acrylate, 2-pentyl (meth)acrylate, 3-pentyl (meth)acrylate, isopentyl acrylate, neopentyl acrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arrachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate, phenoxyethyl (meth)acrylate, t-butylcyclohexyl (meth)acrylate, cyclohexyl (meth)acrylate, ureido (meth)acrylate, tetrahydrofurfuryl (meth)acrylate and mixtures thereof.

3. The cosmetic preparation according to claim 2, where component a) is chosen from the group consisting of tert-butyl acrylate, methyl methacrylate, ethyl methacrylate, n-propyl methacrylate, isopropyl methacrylate, n-butyl methacrylate, tert-butyl methacrylate, isobutyl methacrylate, sec-butyl methacrylate and mixtures thereof.

4. The cosmetic preparation according to claim 1, where component b) is chosen from monoolefinically unsaturatated carboxylic acids and salts thereof.

5. The cosmetic preparation according to claim 4, where component b) is chosen from the group consisting of acrylic acid, methacrylic acid, ethacrylic acid, alpha-chloroacrylic acid, crotonic acid, maleic acid, maleic anhydride, itaconic acid, citraconic acid, mesaconic acid, glutaconic acid, aconitic acid, fumaric acid, half-esters of monoethylenically unsaturated dicarboxylic acids having 4 to 10 carbon atoms.

6. The cosmetic preparation according to claim 5, where component b) comprises or consists of compounds chosen from the group consisting of acrylic acid, methacrylic acid and mixtures thereof.

7. The cosmetic preparation according to claim 5, where component b) comprises or consists of compounds chosen from the group consisting of acrylic acid, itaconic acid and mixtures thereof.

8. The cosmetic preparation according to claim 1, where compounds c1) and/or c2) or mixtures of compounds c1) and/or c2) are used as component c), where the average number of olefinic, free-radically polymerizable double bonds per molecule is more than 2.

9. The cosmetic preparation according to claim 1, where component c) has a molecular weight M_{w} of at least 400 g/mol.

10. The cosmetic preparation according to claim 1, where the at least one polymer A comprises, in copolymerized form,
a) 60-80% by weight of component a),
b) 12-39% by weight of component b),
c) 1-8% by weight of component c) and
d) 0-30% by weight of component d)
with the proviso that the amounts of components a) to d) add up to 100% by weight.

11. The cosmetic preparation according to claim 1, where the preparation also has water and at least one cosmetically acceptable carrier B) which is chosen from
i) water-miscible organic solvents,
ii) oils, fats, waxes,
iii) esters of C₆-C₃₀-monocarboxylic acids with mono-, di- or trihydric alcohols which are different from ii),
iv) saturated acyclic and cyclic hydrocarbons,
v) fatty acids,
vi) fatty alcohols,
vii) propellants (propellant gases) and
viii) mixtures thereof.

12. The cosmetic preparation according to claim 1, in the form of a spray product, where the preparation is present either in combination with a mechanical pump spray device or in combination with at least one propellant chosen from the group consisting of propane, butane, dimethyl ether, fluorinated hydrocarbons and mixtures thereof.

13. A polymer A as defined in claim 1.

14. A method of producing a cosmetic preparation comprising utilizing the polymer A according to claim 13 in the preparation of the cosmetic preparation.

15. The cosmetic preparation according to claim 2, where component b) is chosen from monoolefinically unsaturated carboxylic acids and salts thereof.

16. The cosmetic preparation according to claim 3, where component b) is chosen from monoolefinically unsaturated carboxylic acids and salts thereof.

17. The cosmetic preparation according to claim 5, wherein the half-esters of monoethylenlcally unsaturated dicarboxylic acids have 4-6 carbon atoms.

18. The cosmetic preparation according to claim 1, where component c) has a molecular weight Mw of more than 700 g/mol.

19. The cosmetic preparation according to claim 11, where the at least one cosmetically acceptable carrier B) comprises at least one water-miscible organic solvent and the water-miscible organic solvent is a C₂-C₄ alkanol.

20. The cosmetic preparation according to claim 19, where the C₂-C₄ alkanol is ethanol.
